SEQUENCE LISTING (9 sequences)

SEQ ID NO: 1 - protein, 906 aa, Cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 2 - protein, 719 aa, Cytomegalovirus
TSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNP

SEQ ID NO: 3 - protein, 135 aa, Cytomegalovirus
TRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 4 - protein, 825 aa, synthetic construct
  REGION 1..825: note = Synthetic polypeptide
MEFGLSWLFLVAILKGVQCSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHQTTQSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 5 - protein, 669 aa, synthetic construct
TSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHQTTQSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKV

SEQ ID NO: 6 - protein, 130 aa, synthetic construct
LCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 7 - protein, 26 aa, Homo sapiens
MEFGLSWLFLVAILKGVQCSSSTSHA

SEQ ID NO: 8 - protein, 19 aa, Homo sapiens
MEFGLSWVFLVALFRGVQC

SEQ ID NO: 9 - DNA, 2481 bp, synthetic construct
  misc_feature 1..2481: note = Synthetic polynucleotide
  source 1..2481: mol_type = other DNA
atggagtttgggctgagctggctttttcttgtggctattttaaaaggtgtccagtgttcctcttctacatcccatgccacctcctccacacataacggctctcacacctccaggaccacctctgctcagacaaggtccgtgtactctcagcatgtgacctcctctgaggctgtgtcccaccgggccaacgagaccatctacaacaccaccctgaaatatggagatgtcgtcggcgtgaacaccaccaaatacccctatagggtgtgctccatggcccagggaaccgacctgatcaggttcgagaggaatatcatctgcacctccatgaagcctattaacgaagacctggacgaaggcatcatggtggtgtataagcggaacatcgtcgcccataccttcaaggtgcgggtctaccaaaaggtcctgaccttccggaggtcctacgcctacatttacaccacatatctgctgggctccaacacagaatacgtcgctccccccatgtgggagatccatcacatcaacaagtttgcccaatgctactcttcttactctcgggtcatcggcggaaccgtgttcgtggcttaccatcgggactcctacgagaacaagaccatgcagctgatccccgacgattactccaatacacattccacccggtatgtgaccgtgaaggaccagtggcactctaggggctccacctggctctacagggagacctgtaacctgaactgcatgctcacaatcacaaccgccaggtctaagtacccctatcacttcttcgccacctccaccggagacgtggtctacatttcccccttctacaacggcaccaacaggaacgcctcttacttcggagaaaacgccgacaagttctttatctttcctaactataccattgtgtctgacttcggcaggcccaatgccgctcctgagacacataggctcgtcgctttcctggagagggccgattctgtcatttcctgggacatccaggacgagaagaacgtcacctgccagctgaccttttgggaggcctccgagcggacaatcaggtccgaggccgaggactcctaccatttttcttctgccaagatgaccgccaccttcctgtctaagaaacaagaagtgaacatgtctgattctgccctcgattgcgtgcgggatgaggccattaacaagctccagcagatcttcaacacctcttacaatcagacctacgaaaagtacggaaacgtctctgtgttcgagacctccggcggactggtcgtgttctggcagggcattaaacagaagtctctcgtcgaactcgagcggctcgccaacaggtcttccctgaacatcacccaccaaaccacccagtccacctctgacaacaacaccacacatctctcctccatggagtccgtgcataacctcgtgtatgcccaactccagttcacctacgataccctcaggggctacatcaacagggccctcgctcagatcgccgaagcttggtgcgtggaccagaggaggaccctggaggtcttcaaggagctctccaagatcaacccttccgccatcctgtccgccatctataacaaacccatcgctgctaggttcatgggagatgtgctgggcctcgcctcttgcgtcaccatcaatcaaacctctgtgaaggtcctgagggatatgaacgtcaaagagtctcccggcaggtgttactctaggcctgtggtcatcttcaacttcgctaattcctcctacgtccagtatggccagctgggcgaggacaacgagattctcctgggaaatcaccggaccgaggagtgccagctgccttctctcaagatcttcatcgctggcaactctgcttatgagtatgtggactatctctttaagaggatgatcgacctgtcttctatctctaccgtcgactccatgatcgccctggatatcgaccccctggagaataccgacttcagggtgctcgagctgtattcccagaaggaactgcggtcctccaatgtcttcgacctggaggaaatcatgcgggagttcaactcctacaaacagagggtgaagtacgtggaggacaaggtcctgtgcacacagcccctgcagaatctgtttccctatctggtgtccgccgacggaaccacagtgacatccggctctacaaaggacacctccctgcaggcccccccttcctacgaggagtccgtctacaactctggaaggaaaggacctggacccccttcttctgatgcctctacagccgctcctccttacaccaacgagcaggcttaccagatgctgctcgccctggctcggctcgatgctgagcagagggctcaacagaatggcacagactctctggatggccagaccggcacccaagacaagggccagaaacccaacctcctggatcggctgaggcatcggaagaacggctacaggcacctcaaagattctgacgaagaggagaacgtgtgatga

(19) United States
(12) Patent Application Publication
(10) Pub. No.: US 2025/0134990 A1
(43) Pub. Date: May 1, 2025

(54) IMMUNOGENIC COMPOSITIONS AND USES THEREOF

(71) Applicant: The Council of the Queensland Institute of Medical Research, Herston, Queensland (AU)

(72) Inventors: Rajiv KHANNA, Herston (AU); Vijayendra DASARI, Herston (AU)

(21) Appl. No.: 18/692,605

(22) PCT Filed: Sep. 16, 2022
(86) PCT No.: PCT/AU2022/051120

§ 371 Date: Mar. 15, 2024

(30) Foreign Application Priority Data
Sep. 16, 2021 (AU) 2021902988

(51) Int. Cl. A61K 39/245; A61P 37/04; C07K 14/005

(52) U.S. Cl. CPC A61K 39/245; A61P 37/04; C07K 14/005; C07K 2319/02; C07K 2319/036; C07K 2319/40; C12N 2710/16122; C12N 2710/16134; C12N 2710/16171

(57) ABSTRACT

The present invention discloses a novel recombinant polypeptide, and methods for stimulating protective or therapeutic immune responses to human herpesvirus. The invention relates to a composition comprising isolated homotrimers of a modified gB polypeptide from human cytomegalovirus (hCMV), wherein the gB polypeptide comprises an immunoglobulin signal peptide, an ex-tracellular domain with furin cleavage site mutations, and/or the intravirion domain, but lacks the transmembrane domain. Also the use thereof and methods of treating and preventing CMV associated disease or CMV infections with said composition.

### RELATED APPLICATIONS

This application claims priority to Australian Provisional Application No. 2021902988 entitled “Immunogenic Compositions And Uses Thereof” filed 16 Sep. 2021, the contents of which are incorporated herein by reference in their entirety.

### FIELD OF THE INVENTION

This invention relates generally to compositions and methods for stimulating immune responses. More particularly, the present invention relates to a novel recombinant polypeptide, and methods for stimulating protective or therapeutic immune responses to human herpesvirus.

### BACKGROUND OF THE INVENTION

Primary human cytomegalovirus (HCMV) in healthy individuals is generally asymptomatic, establishing a latent state with occasional reactivation and shedding from mucosal surfaces. In some cases, primary HCMV infection is accompanied with clinical symptoms of a mononucleosis-like illness, similar to that caused by Epstein-Barr virus. There are two important clinical settings where HCMV causes significant morbidity and mortality. These include congenital primary infection and primary or reactivation of the virus in immunosuppressed adults.

The HCMV envelope glycoprotein B (gB) protein is a highly conserved glycoprotein and plays an important role in mediating virus-host cell fusion virus entry into all cell types. The HCMV gB interacts with other HCMV envelope proteins, such as gH, gL, gO and UL128/UL130/UL131A during HCMV fusion and entry process into host cells. The native conformation of the HCMV gB protein exists as a trimer which subsequently dimerises within the viral envelope. The HCMV gB protein is a highly immunogenic antigen, as gB-specific antibodies can be detected in all naturally infected individuals. Therefore, the gB protein is considered as a major target antigen for vaccine development.

The most extensively tested HCMV vaccine formulation in various phase II clinical trials contains a modification where the furin cleavage site is removed, as retention of the proteolytic cleavage site interfered with recombinant protein production (Spaete, Transplant Proc., 1991). Such production methods resulted in a monomeric soluble form of the gB protein. Notably this gB protein construct with MF59 adjuvant demonstrated only a 50% efficacy in preventing HCMV infection in solid organ transplant recipients (Pass et al., N. Enl. J Med., 2009; and Pass, J. Clin Virol., 2009).

Because the natural form of HCMV gB protein is the trimer, this is proposed to be the optimal physical structure for eliciting neutralising antibody (Fu et al., Vaccine, 2014). Recent studies have described production of a fully trimeric recombinant HCMV gB protein, that elicits markedly higher titres of serum HCMV neutralising antibodies in mice relative to its monomeric counterpart (Cui et al., Vaccine, 2018). Production of this trimeric recombinant protein saw the furin cleavage site replaced with a 15 amino acid (Gly₄Ser) 3 linker sequence, as well as a 6×His sequence added to the 3′ end to better enable protein purification.

### SUMMARY OF THE INVENTION

The present invention is predicated, at least in part, in the inventor's identification that a modified gB polypeptide that multimerises into trimers was produced by removing at least a portion of the transmembrane domain of the native full-length gB protein. These modified gB polypeptides elicit a substantial immune response, and as such have clear clinical utility in the treatment and/or prevention of CMV infection and/or CMV-associated disease.

Accordingly, in one aspect the present invention provides a composition comprising isolated homotrimers of a modified gB polypeptide. Suitably, the modified gB polypeptide comprises an amino acid sequence corresponding to a human cytomegalovirus (HCMV) gB protein, wherein the amino acid sequence lacks at least a portion of the transmembrane region.

In some embodiments, the transmembrane domain corresponds to amino acid residues 751 to 771 of the native full-length polypeptide sequence set forth in SEQ ID NO: 1. In some preferred embodiments, the modified gB polypeptide amino acid sequence slacks substantially of the of transmembrane region.

In some embodiments, the modified gB polypeptide comprises a first region that corresponds to at least a portion of the gB protein virion surface domain; and a second region that corresponds to at least a portion of the gB protein intravirion domain. Typically, the virion surface domain comprises amino acid residues 32 to 705 of the sequence set forth in SEQ ID NO: 1. In some embodiments, the intravirion domain comprises amino acid residues 772 to 906 of the sequence set forth in SEQ ID NO: 1.

In some embodiments, the modified gB polypeptide further comprises an N-terminal signal peptide. In some embodiments, the signal peptide facilitates the secretion of the modified gB polypeptide from a cell. In some embodiments, the signal peptide is derived from an immunoglobulin isotype. In some embodiments of this type, the immunoglobulin isotype is selected from any one of IgA, IgD, IgE, IgG, and IgM. In some preferred embodiments, the signal peptide comprises, consists, or consists of the amino acid sequence set forth in SEQ ID NO: 7.

In some embodiments, the modified gB polypeptide does not include a furin cleavage site motif. In some embodiments of this type, the amino acid residue corresponding to position 456 of the wild-type HCMV gB protein (i.e., the sequence set forth in SEQ ID NO: 1) is an amino acid other than arginine. Typically, the amino acid residue corresponding to position 456 of the wild-type HCMV gB protein is glutamine or threonine. In some of the same embodiments and other some embodiments, the amino acid residue corresponding to position 458 of the wild-type HCMV gB protein is an amino acid other than arginine. Typically, the amino acid residue corresponding to position 458 of the wild-type HCMV gB protein is threonine or glutamine. In some of the same embodiments and some other embodiments, the amino acid residue corresponding to position 459 of the wild-type HCMV gB protein is an amino acid other than arginine. Typically, the amino acid residue corresponding to position 459 of the wild-type HCMV gB protein is glutamine or threonine.

In some embodiments, the modified gB polypeptide homotrimers dimerize to form a hexamer.

In some embodiments, the modified gB polypeptide complexes are present in a prefusion confirmation.

In another aspect, the present invention provides a nucleic acid molecule that encodes a modified gB polypeptide comprising, consisting, or consisting essentially of an amino acid sequence that corresponds to a human cytomegalovirus (HCMV) gB protein, wherein the amino acid sequence lacks at least a portion of the transmembrane region.

In yet another aspect, the present invention provides an expression vector that includes the nucleic acid molecules described above and/or elsewhere herein, operably connected to a regulatory element. In some embodiments, the regulatory element is a promotor.

In still yet another aspect, the present invention provides a cell that comprises the expression vector described above and/or elsewhere herein.

In another aspect the present invention provides a pharmaceutical composition comprising a preparation that comprises, consists, or consists essentially of an amino acid sequence that corresponds to a HCMV envelope gB protein, wherein the amino acid sequence lacks at least a portion of the transmembrane region; and a pharmaceutically acceptable carrier, excipient, and/or diluent. In some embodiments, the pharmaceutical composition may further comprise an adjuvant.

In some embodiments, the transmembrane domain corresponds to amino acid residues 751 to 771 of the native full-length polypeptide sequence set forth in SEQ ID NO: 1.

In some embodiments, the modified polypeptide further comprises a N-terminal signal peptide. In some embodiments, the signal peptide facilitates the secretion of the polypeptide from a cell.

In some embodiments, the signal peptide is derived from an immunoglobulin isotype. In some embodiments of this type, the immunoglobulin isotype is selected from any one of IgA, IgD, IgE, IgG, and IgM. In some preferred embodiments, the signal peptide comprises, consists, or consists of the amino acid sequence set forth in SEQ ID NO: 7.

In some embodiments, the polypeptide comprises a first region that corresponds to at least a portion of the gB virion surface domain; and a second region that corresponds to at least a portion of the gB intravirion domain. Typically, the gB virion surface domain comprises amino acid residues 32 to 705 of the sequence set forth in SEQ ID NO: 1. In some embodiments, the gB intravirion domain comprises amino acid residues 772 to 906 of the sequence set forth in SEQ ID NO: 1.

In some embodiments, the polypeptide does not include a furin cleavage site motif. In some embodiments of this type, the amino acid residue corresponding to position 456 of the wild-type HCMV gB protein (i.e., the sequence set forth in SEQ ID NO: 1) is an amino acid other than arginine. Typically, the amino acid residue corresponding to position 456 of the wild-type HCMV gB protein is glutamine.

In some of the same embodiments and other embodiments, the amino acid residue corresponding to position 458 of the wild-type HCMV gB protein is an amino acid other than arginine. Typically, the amino acid residue corresponding to position 458 of the wild-type HCMV gB protein is threonine.

In some of the same embodiments and other embodiments, the amino acid residue corresponding to position 459 of the wild-type HCMV gB protein is an amino acid other than arginine. Typically, the amino acid residue corresponding to position 459 of the wild-type HCMV gB protein is glutamine.

In some embodiments, the pharmaceutical composition comprises a modified polypeptide comprising, consisting, or consisting essentially of, the amino acid sequence set forth in SEQ ID NO: 2.

In some embodiments, the polypeptide complexes to form a multimer.

In still yet another aspect, the present invention provides a method for treating or preventing a CMV infection and/or a CMV-associate disease or condition in a subject, the method comprising administering to the subject a composition comprising a modified polypeptide that comprises, consists, or consists essentially of an amino acid sequence that corresponds to a HCMV gB protein, wherein the amino acid sequence lacks at least a portion of the transmembrane region. In some embodiments of this type, the method further comprises administering one or more ancillary agents to the subject.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 provides a flow diagram of HCMV gB protein expression and purification process.

FIG. 2 provides a graphical representation of the modified gB polypeptide expression and purification. The HCMV gB protein encoding sequence was codon optimised for mammalian expression and then cloned into a mammalian expression vector. (A) Non-reducing SDS-PAGE analysis of the modified gB polypeptide expressed in 15 L fermenter. (B) Non-reducing SDS-PAGE analysis of anion exchange chromatography. (C) Non-reducing SDS-PAGE analysis of CHT Type II chromatography. (D) Non-reducing SDS-PAGE analysis of cation exchange chromatography and purified modified gB polypeptide in its multimeric form. M: molecular weight marker; R: reference standard; CP: clarified and concentrated protein; L: load; FT: flow through; W: wash; E: elution; and S: column strip fraction.

FIG. 3 provides graphical representations of the modified polypeptide purification and characterisation. (A) modified gB polypeptide size exclusion chromatography. Purified modified gB polypeptide was concentrated to 20 mg/ml and then 0.5 mL of protein was loaded onto a SUPERDEX 200 Increase 10/300 GL column. Protein was then eluted with 25 mM Tris 500 mM NaCl (pH 7.2) buffer. (B) The modified gB polypeptide purified on SUPERDEX column was analysed on 8% native PAGE-SDS PAGE gel. Protein samples were loaded without DDT and samples were not boiled. Lane 1: molecular weight marker; lane 2: modified gB polypeptide in 25 mM glycine 500 mM NaCl (pH 4.0) SUPERDEX load; and lanes 3 to 9: modified gB polypeptide SUPERDEX purified fractions E5 to E11.

FIG. 4 provides a graphical representation of HCMV gB protein specific antibody responses in human HLA A24 transgenic mice. (A) Two groups of human HLA A24 transgenic mice were immunised subcutaneously three times (day 0, 21 and 42) with Group 1: CMV vaccine (n=6) formulated with modified gB polypeptide (5 ug), CMVpoly (30 ug) and CpG1018 (50 ug); or Group 2: control formulation (n=4) CpG1018 (50 ug). On day 49 mice were sacrificed and serum samples were collected to analyse HCMV gB-specific antibody responses. (B) Line graph represents the total HCMV gB-specific immunoglobulin titres induced after immunisation of mice with three isoforms of gB polypeptide. VM1 to VM6 represents the vaccine group mice and CM1 to CM4 represents the control group mice. (C) Western blot analysis of HCMV gB polypeptide using two different concentrations (1:1000 and 1:3000) of mouse serum obtained after immunisation with CMV vaccine under non-reducing condition.

FIG. 5 provides characterisation of HCMV gB specific antibody responses. Two groups of human HLA A24 transgenic mice were immunised subcutaneously three times (day 0, 21 and 42) with CMV vaccine (n=6) formulated with gB polypeptide (5 μg), CMVpoly (30 μg) and CpG1018 (50 μg); or control formulation (n=4) CpG1018 (50 μg). On day 49 mice were sacrificed and serum samples were collected to analyse HCMV gB-specific antibody responses. (A) Line graph represents HCMV gB-specific immunoglobulin isotypes, IgA, IgM, IgG1, IgG2a, IgG2b and IgG3 induced after immunisation with CMV vaccine. (B) Bar graph represents 50% neutralising antibody titres against Mrc-5 cells infected with HCMV AD169 strain and ARPE-19 cells infected with HCMV TB40e strain. (C and D) Mrc-5 cells were infected with AD169 strain overnight. Serum obtained from mice vaccinated with CpG1018 alone or CMV vaccine was diluted (1:512 and 1:1024) and then added to Mrc-5 cells infected with HCMV AD169 strain. Cells were stained with anti-mouse Ig antibody conjugated to FITC. The frequency of mouse antibodies binding to Mrc-5 cells infected with HCMV AD169 strain determined by flow cytometry analysis. (C) Bar graph represents the frequency of HCMV gB-specific antibodies binding to Mrc-5 cells infected with HCMV AD169 strain. (D) Representative FACS plots.

FIG. 6 provides characterisation of HCMV gB specific B cell responses. Two groups of human HLA A24 transgenic mice were immunised subcutaneously three times (day 0, 21 and 42) with CMV vaccine (n=6) formulated with modified gB polypeptide (5 μg), CMVpoly (30 μg) and CpG1018 (50 μg) or control formulation (n=4) CpG1018 (50 μg). On day 49 mice were sacrificed and spleens were collected, and single cell suspension were made to analyse T_{FH} cells, GC B cells and gB-specific antibody secreting B cells. (A) Bar graph represents the frequency of CxCr5⁺PD1⁺CD4⁺ T cells (T_{FH} cells). (B) Bar graph represents the frequency of B220⁺GL7⁺FAS⁺ B cells (GC B cells). (C and D) Bar graph and ELISpot well picture represents the frequency of plasma and memory B cells secreting gB-specific antibodies. Error bars represent the mean±SEM *, p<0.05; **, p<0.01 (determined by student t test).

FIG. 7 provides characterisation of HCMV gB specific T cell responses. Two groups of human HLA A24 transgenic mice were immunised subcutaneously three times (day 0, 21 and 42) with CMV vaccine (n=6) formulated with the modified gB polypeptide (5 μg), CMVpoly (30 μg) and CpG1018 (50 μg) or control formulation (n=4) CpG1018 (50 μg). On day 49 mice were sacrificed and spleens were collected, and single cell suspension were made to analyse HCMV gB-specific CD4⁺ T cell responses. To measure the HCMV gB-specific CD4⁺ T cells responses, splenocytes were stimulated with gB pepmix and then measured their ability to secrete multiple cytokines (IFN-γ, TNF and IL-2) using intracellular cytokine staining (ICS). To further expand HCMV gB-specific CD4+ T cells, splenocytes from control and CMV vaccine groups were in vitro stimulated with gB pepmix and then cultured for 10 days. In vitro expanded gB-specific CD4⁺ T cells were assessed for their ability to secrete multiple cytokines (IFN-γ, TNF and IL-2) using ICS. (A) Represents the mean frequencies of HCMV gB-specific CD4⁺ T cells producing IFN-γ ex vivo. (B) Pie chart shows gB-specific CD4⁺ T cells secreting different combinations of IFN-γ, TNF and IL-2 ex vivo. (C) Represents the mean frequencies of HCMV gB-specific CD4⁺ T cells producing IFN-γ after in vitro expansion. (D) Pie chart shows gB-specific CD4⁺ T cells secreting different combinations of IFN-γ, TNF and IL-2 after in vitro expansion. Error bars represents the mean±SEM *, p<0.05; (determined by student t test).

FIG. 8 provides a photographical representation of the modified gB polypeptide homotrimers, as obtained by cryogenic electron microscopy.

TABLE 1
BRIEF DESCRIPTION OF THE SEQUENCES

| SEQ ID | | |
|---|---|---|
| No | SEQUENCE DESCRIPTION | TYPE |
| 1 | Native HCMV gB protein from HCMV strain | aa |
| | AD169 | |
| 2 | Native HCMV gB virion surface domain | aa |
| 3 | Native HCMV gB intravirion domain | aa |
| 4 | Modified gB polypeptide | aa |
| 5 | Modified polypeptide virion surface domain | aa |
| 6 | Modified polypeptide intravirion domain | aa |
| 7 | IgG heavy chain signal peptide | aa |
| 8 | IgG heavy chain signal peptide | aa |
| 9 | Polynucleotide sequence encoding modified | nt |
| | gB polypeptide | |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

As used herein, the indefinite articles ‘a’ and ‘an’ are used here to refer to or encompass singular or plural elements or features and should not be taken as meaning or defining “one” or a “single” element or feature. For example, “a” protein includes one protein, one or more proteins or a plurality of proteins.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time (e.g., one agent is administered within from about 1 min to within about 1 hour) before or after another. Any contemporaneous time is useful. However, it will often be the case that when not administered simultaneously, the agents will be administered within about 1 min to within about 8 hours and preferably within less than about 1 hour to about 4 hours. When administered contemporaneously, the agents are suitably administered at the same site on the subject. The term “same site” includes the exact location, but can be within about 0.5 cm to about 15 cm, preferably from within about 0.5 cm to about 5 cm. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

As used herein, “and/or” refers to and encompasses any and all possible combinations of one or more of the associated listed items, as well as the lack of combinations when interpreted in the alternative (or).

By “coding sequence” is meant any nucleic acid sequence that contributes to the code for the polypeptide product of a gene or for the final mRNA product of a gene (e.g., the mRNA product of a gene following splicing). By contrast, the term “non-coding sequence” refers to any nucleic acid sequence that does not contribute to the code for the polypeptide product of a gene or for the final mRNA product of a gene.

Unless the context requires otherwise, the terms “comprise”, “comprises” and “comprising”, or similar terms are intended to mean a non-exclusive inclusion, such that a recited list of elements or features does not include those stated or listed elements solely but may include other elements or features that are not listed or stated.

By “consisting essentially of” in the context of an amino acid sequence, such as an isolated protein, is meant the recited amino acid sequence together with an additional one, two or three amino acids at the N- or C-terminus.

The terms “construct” and “synthetic construct” are used interchangeably herein to refer to heterologous nucleic acid sequences that are operably linked to each other and may include sequences providing the expression of a polynucleotide in a host cell and optionally sequences that provide for the maintenance of the construct.

By “corresponds to” or “corresponding to” is meant an antigen which encodes an amino acid sequence that displays substantial sequence identity or similarity to an amino acid sequence in a target antigen. In general, the antigen will display at least about 30, 40, 50, 55, 60, 65, 70, 75, 80, 85, 90, 91, 92, 93, 94, 95, 96, 97, 98, 99% identity or similarity to at least a portion of the target antigen.

By “effective amount”, in the context of stimulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individuals to be treated, the formulation of the composition, the assessment of the medical situation, and other relevant factors. It is expected that the amount will fall in a relatively broad range that can be determined through routine trials.

By “expression vector” is meant any autonomous genetic element capable of directing the synthesis of a protein encoded by the vector. Such expression vectors are known by practitioners in the art.

The terms “gB” and “gB protein” and the like, as used herein means “glycoprotein B”, a polypeptide having a sequence according to UniProt accession no. P06473, the product of a gB gene (e.g., the HCMV gB gene (identified by GenBank accession no. 3077424)), and includes all of the variants, isoforms and virus homologues of gB.

The term “gene” is used in its broadest context to include both a genomic DNA region corresponding to the gene as well as a cDNA sequence corresponding to exons or a recombinant molecule engineered to encode a functional form of a product.

To enhance immune response (“immunoenhancement”), as is well-known in the art, means to increase the animal's capacity to respond to foreign or disease-specific antigens (e.g., virus antigens) i.e., those cells primed to attack such antigens are increased in number, activity, and ability to detect and destroy those antigens. Strength of immune response is measured by standard tests including: direct measurement of peripheral blood lymphocytes by means known to the art; natural killer cell cytotoxicity assays (see, e.g., Provinciali M. et al. (1992, J. Immunol. Meth. 155:19-24), cell proliferation assays (see, e.g., Vollenweider, I. and Groseurth, P. J. (1992, J. Immunol. Meth. 149:133-135), immunoassays of immune cells and subsets (see, e.g., Loeffler, D. A., et al. (1992, Cytom. 13:169-174); Rivoltini, L., et al. (1992, Can. Immunol. Immunother. 34:241-251); or skin tests for cell-mediated immunity (see, e.g., Chang, A. E. et al. (1993, Cancer Res. 53:1043-1050). Any statistically significant increase in strength of immune response as measured by the foregoing tests is considered “enhanced immune response”, “immunoenhancement” or “immunopotentiation” as used herein. Enhanced immune response is also indicated by physical manifestations such as fever and inflammation, as well as healing of systemic and local infections, and reduction of symptoms in disease, i.e., decrease in virus load, alleviation of symptoms of a disease or condition including, but not restricted to, a CMV-associated disease or condition. Such physical manifestations also define “enhanced immune response” “immunoenhancement” or “immunopotentiation” as used herein.

By “isolated” is meant material that is substantially or essentially free from components that normally accompany it in its native state.

A composition is “immunostimulatory” if it is capable of either: a) generating an immune response against an antigen (e.g., a virus antigen) in a naive individual; or b) reconstituting, boosting, or maintaining an immune response in an individual beyond what would occur if the compound or composition was not administered. A composition is immunogenic if it is capable of attaining either of these criteria when administered in single or multiple doses.

As used herein, “preventing” (or “prevent” or “prevention”) refers to a course of action (such as administering a pharmaceutical composition of the present invention) initiated prior to the onset of a symptom, aspect, or characteristic of a CMV infection or a CMV-associated disease, disorder or condition, so as to prevent or reduce the symptom, aspect, or characteristic. It is to be understood that such preventing need not be absolute to be beneficial to a subject. A “prophylactic” treatment is a treatment administered to a subject who does not exhibit signs of a CMV infection or a CMV-associated disease, disorder or condition, or exhibits only early signs for the purpose of decreasing the risk of developing a symptom, aspect, or characteristic of a CMV infection or a CMV-associated disease, disorder or condition. By “stimulating” is meant directly or indirectly increasing the level and/or functional activity of a target molecule. For example, an agent may indirectly stimulate the said level/activity by interacting with a molecule other than the target molecule. In this regard, indirect stimulation of a gene encoding a target polypeptide includes within its scope stimulation of the expression of a first nucleic acid molecule, wherein an expression product of the first nucleic acid molecule stimulates the expression of a nucleic acid molecule encoding the target polypeptide. In certain embodiments, “stimulation” or “stimulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than if the antigen had been used alone.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys, and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software.

“Similarity” refers to the percentage number of amino acids that are identical or constitute conservative substitutions as defined in Table 2.

TABLE 2
AMINO ACID SUBSTITUTIONS

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Similarity may be determined using sequence comparison programs such as GAP (Deveraux et al., 1984. Nucleic Acids Res. 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polypeptides or polynucleotides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997. Nucleic Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As defined herein the term “signal peptides” (also referred to as leader peptide, targeting signal, signal sequence, transit peptide or localisation signal) are sequence motifs targeting proteins for translocation across the endoplasmic reticulum membrane. Signal peptides are found at the amino terminus of nascent proteins, and function by prompting the transport mechanism within the cell to bring the proteins to their specific destination within the cell, or outside the cell if the proteins are to be secreted. If secreted in the extracellular environment, it may be specified that the signal peptide are secretory signal peptides.

As used herein, “treating” (or “treat” or “treatment”) refers to a therapeutic intervention that ameliorates a sign or symptom of a CMV infection, inclusive of a CMV-associated disease, disorder or condition, after it has begun to develop. The term “ameliorating,” with reference to a CMV-associated disease, disorder or condition, refers to any observable beneficial effect of the treatment. Treatment need not be absolute to be beneficial to the subject. The beneficial effect can be determined using any methods or standards known to the ordinarily skilled artisan.

In the context of the present invention, by “CMV-associated disease, disorder or condition” is meant any CMV infection, inclusive of any clinical pathology resulting from such an infection by a cytomegalovirus, such as those hereinbefore described.

By “administering” or “administration” is meant the introduction of a composition disclosed herein into a subject by a particular chosen route. Any safe route of administration and dosage form, such as those hereinbefore described, may be employed for providing a patient with the composition of the invention.

As generally used herein, the terms “patient”, “individual” and “subject” are used in the context of any mammalian recipient of a treatment or composition disclosed herein. Accordingly, the methods and compositions disclosed herein may have medical and/or veterinary applications. In a preferred form, the subject is a human.

The term “nucleic acid” or “polynucleotide” as used herein designates single- or double-stranded mRNA, RNA, CRNA, RNAi, SIRNA and DNA inclusive of cDNA, mitochondrial DNA (mtDNA) and genomic DNA.

“Polypeptide,” “peptide” and “protein” are used interchangeably herein to refer to a polymer of amino acid residues and to variants and synthetic analogues of the same. As used herein, the terms “polypeptide,” “peptide” and “protein” are not limited to a minimum length of the product. Thus, peptides, oligopeptides, dimers, multimers, and the like, are included within the definition. Both full-length proteins and portions thereof are encompassed by the definition. The terms “biologically active portions” or “fragments” are used interchangeably herein, to describe an immunogenic portion of a HCMV gB polypeptide. These portions can be a polypeptide which is, for example, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or more amino acid residues in length. The isolated proteins described herein, inclusive of fragments, variants and derivatives thereof, may be produced by any means known in the art, including but not limited to, chemical synthesis, recombinant DNA technology and proteolytic cleavage to produce peptide fragments.

Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6. Typically, recombinant protein preparation includes expression of a nucleic acid encoding the protein in a suitable host cell.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector (i.e., a vector that exists as an extrachromosomal entity) the replication of which is independent of chromosomal replication, (e.g., a linear or closed circular plasmid), an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

The term “wild-type”, with respect to an organism, polypeptide, or nucleic acid sequence, refers to an organism, polypeptide or nucleic acid sequence that is naturally occurring or available in at least one naturally occurring organism which is not changed, mutated, or otherwise manipulated by man.

In order that the invention may be readily understood and put into practical effect, particular preferred embodiments will now be described by way of the following non-limiting experimental examples.

### 2. Compositions

The present invention is based, at least in part, on the determination that a recombinantly produced modified gB polypeptide that lacks at least a portion of the transmembrane domain is capable of stimulating or eliciting enhanced immune responses to CMV, as compared to the recombinantly produced native gB protein. The present inventors determined that this modified gB polypeptide would be effective as a preventative and/or therapeutic treatment for CMV infection. In doing so, compositions that comprise the polypeptide are also effective at preventing or prophylactically or therapeutically treating a CMV-associated disease, disorder or condition. Accordingly, the present invention provides modified gB polypeptides with an amino acid sequence that corresponds to the native HCMV gB polypeptide, but lacking at least a portion of the transmembrane domain, in compositions and methods for treating or preventing CMV infection or CMV-associated disease, disorders or conditions in a subject.

### 2.1 Modified gB Polypeptide

HCMV cell entry requires the conserved gB protein, which functions as a fusogen and is reported to bind signalling receptors. gB protein elicits a strong immune response in humans and induces the production of neutralising antibodies, although most anti-gB protein antibodies are non-neutralising. Viral fusogens mediate the merger of the viral envelope and host membrane during virus entry and cell spread by undergoing a series of conformational changes from the prefusion to the post-fusion form mapped out for several viral fusogens.

The gB protein is encoded by the UL55 gene. Sequence variation of the UL55 gene indicates there are four major gB genotypes (gB1, gB2, gB3, gB4). In addition, three non-prototypic genotypes (gB5, gB6, and gB7) have also been identified. The full length native HCMV gB protein is 906 amino acids in length and contains a signal sequence, virion domain (i.e., an ectodomain) which includes a hydrophobic membrane proximal region, a transmembrane domain, and the intraviral (or cytoplasmic) domain (cytodomain).

The full-length native gB protein amino acid sequence, as deposited under UniProt accession No. P06473, is set forth below.

[SEQ ID NO: 1]
MESRIWCLVVCVNLCIVCLGAAVSSSSTSHATSSTHNGSHTSRTTSAQT
RSVYSQHVTSSEAVSHRANETIYNTTLKYGDVVGVNTTKYPYRVCSMAQ
GTDLIRFERNIICTSMKPINEDLDEGIMVVYKRNIVAHTFKVRVYQKVL
TFRRSYAYIYTTYLLGSNTEYVAPPMWEIHHINKFAQCYSSYSRVIGGT
VFVAYHRDSYENKTMQLIPDDYSNTHSTRYVTVKDQWHSRGSTWLYRET
CNLNCMLTITTARSKYPYHFFATSTGDVVYISPFYNGTNRNASYFGENA
DKFFIFPNYTIVSDFGRPNAAPETHRLVAFLERADSVISWDIQDEKNVT
CQLTFWEASERTIRSEAEDSYHFSSAKMTATFLSKKQEVNMSDSALDCV
RDEAINKLQQIFNTSYNQTYEKYGNVSVFETSGGLVVFWQGIKQKSLVE
LERLANRSSLNITHRTRRSTSDNNTTHLSSMESVHNLVYAQLQFTYDTL
RGYINRALAQIAEAWCVDQRRTLEVFKELSKINPSAILSAIYNKPIAAR
FMGDVLGLASCVTINQTSVKVLRDMNVKESPGRCYSRPVVIFNFANSSY
VQYGQLGEDNEILLGNHRTEECQLPSLKIFIAGNSAYEYVDYLFKRMID
LSSISTVDSMIALDIDPLENTDFRVLELYSQKELRSSNVFDLEEIMREF
NSYKQRVKYVEDKVVDPLPPYLKGLDDLMSGLGAAGKAVGVAIGAVGGA
VASVVEGVATFLKNPFGAFTIILVAIAVVIITYLIYTRQRRLCTQPLQN
LFPYLVSADGTTVTSGSTKDTSLQAPPSYEESVYNSGRKGPGPPSSDAS
TAAPPYTNEQAYQMLLALARLDAEQRAQQNGTDSLDGQTGTQDKGQKPN
LLDRLRHRKNGYRHLKDSDEEENV.

The virion surface domain is a large external domain decorated with N-linked oligosaccharides and corresponds with amino acid residues 32 to 750 of the native full length HCMV gB protein set forth in SEQ ID NO: 1. The virion surface domain amino acid sequence is provided herein as SEQ ID NO: 2. At the C-terminal end of this domain is a hydrophobic membrane proximal region, that comprises amino acids 696 to 750 of the native full length HCMV gB protein set forth in SEQ ID NO: 1. In some preferred embodiments, the modified gB polypeptide does not comprise at least part of the hydrophobic membrane proximal region that is present in the full length native HCMV gB protein. In some preferred embodiments the modified gB polypeptide lacks the hydrophobic membrane proximal region that is present in the full length native HCMV gB protein.

The intravirion domain is a small internal domain that is involved in interactions with internal viral components. The intravirion domain corresponds to amino acid residues 772 to 906 of the full length native HCMV gB protein set forth in SEQ ID NO: 1. The intravirion domain amino acid sequence is provided herein as SEQ ID NO: 3.

The virion surface domain and intravirion domain are linked by a single membrane-spanning domain (i.e., the transmembrane domain), which allows for the gB protein to be inserted through the lipid bilayer. The transmembrane domain corresponds with amino acid residues 751 to 771 of the full-length native HCMV gB protein set forth in SEQ ID NO: 1. In some preferred embodiments of the invention, the modified gB polypeptide sequence lacks at least a portion of the transmembrane domain of the full length native HCMV gB protein. For example, the modified gB polypeptide may lack substantially all of the transmembrane domain.

In some embodiments of this type, the modified gB polypeptide also lacks at least a portion of the hydrophobic membrane proximal region of the full length native HCMV gB protein. For example, in some embodiments, the modified gB polypeptide may lack substantially all of the hydrophobic membrane proximal region of the full length native HCMV gB protein.

The full length native HCMV gB protein contains a furin cleavage site motif at the position corresponding to amino acids 457 to 460 of the sequence identified by SEQ ID NO: 1. The furin protease cleaves the HCMV gB protein into gp90 and gp58 subunits, which are covalently linked by disulfide bonds and the mature glycosylated gB protein obtains a trimeric form. The furin cleavage site motif is a sequence pattern of amino acids that is recognised and cleaved by the proprotein convertase, furin, to convert a protein precursor to functional proteins. Generally, the native cleavage site motif is described as a four amino acid pattern R₄-X₃-[K/R] ₂-R₁*, where * represents where the peptide is cleaved. Position 1 (R1) requires a positively charged arginine (R) residue. A mutation of the arginine at this position diminishes the detectable furin cleavage.

By way of an illustrative example, a suitable modified gB polypeptide comprises a sequence corresponding to the native human gB protein, described above and/or elsewhere herein. More specifically, the modified gB polypeptide lacks at least a portion of the native transmembrane domain (e.g., lacking at least a portion of amino acid residues 751 to 771 of the wild type human gB polypeptide set forth in SEQ ID NO: 1). Accordingly, in some embodiments, the modified gB polypeptide comprises an amino acid sequence that corresponds to at least a portion of the wild-type gB virion surface domain (as set forth in SEQ ID NO: 2) and an amino acid sequence that corresponds to at least a portion of the wild-type gB intravirion domain (as set forth in SEQ ID NO: 3). In some embodiments of this type, the modified gB polypeptide lacks at least a portion of the hydrophobic membrane proximal region (i.e., corresponding to amino acid residues 696 to 750 of the full length native gB protein sequence set forth in SEQ ID NO: 1) of the virion surface domain. Preferably, the modified gB polypeptide lacks a transmembrane domain and the hydrophobic membrane proximal region.

In some embodiments the polypeptide may comprise an amino acid sequence that shares at least 70% (and at least 71% to at least 99% and all integer percentages in between) sequence similarity or sequence identity with one or both of the sequences set forth in SEQ ID NOs: 3 and 4, or a fragment of such polypeptides. In more specific embodiments, the polypeptide may comprise an amino acid sequence that shares at least 75%, 80%, 85%, 90%, 91%, 92%, 93%, 94%, 95%, 96%, 97%, 98% or 99% sequence similarity or sequence identity with one or both of the sequences set forth in SEQ ID NOs: 3 and 4, or a fragment of such polypeptides.

In some embodiments, the portion of the native virion surface domain comprises an N-terminal or C-terminal truncation from the full-length native virion surface domain amino acid sequence. In some preferred embodiments the truncation occurs at the C-terminus of the native virion surface domain. By way of an illustrative example, the modified gB polypeptide may comprise an amino acid sequence corresponding to amino acid residues 1 to 669 of the full-length native virion surface domain (i.e., the amino acid sequence set forth in SEQ ID NO: 3). An exemplary sequence of suitable portion of the virion surface domain is identified in SEQ ID NO: 5, and set forth below.

[SEQ ID NO: 5]
TSSTHNGSHTSRTTSAQTRSVYSQHVTSSEAVSHRANETIYNTTLKYGD
VVGVNTTKYPYRVCSMAQGTDLIRFERNIICTSMKPINEDLDEGIMVVY
KRNIVAHTFKVRVYQKVLTFRRSYAYIYTTYLLGSNTEYVAPPMWEIHH
INKFAQCYSSYSRVIGGTVFVAYHRDSYENKTMQLIPDDYSNTHSTRYV
TVKDQWHSRGSTWLYRETCNLNCMLTITTARSKYPYHFFATSTGDVVYI
SPFYNGTNRNASYFGENADKFFIFPNYTIVSDFGRPNAAPETHRLVAFL
ERADSVISWDIQDEKNVTCQLTFWEASERTIRSEAEDSYHFSSAKMTAT
FLSKKQEVNMSDSALDCVRDEAINKLQQIFNTSYNQTYEKYGNVSVFET
SGGLVVFWQGIKQKSLVELERLANRSSLNITHQTTQSTSDNNTTHLSSM
ESVHNLVYAQLQFTYDTLRGYINRALAQIAEAWCVDQRRTLEVFKELSK
INPSAILSAIYNKPIAARFMGDVLGLASCVTINQTSVKVLRDMNVKESP
GRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQ
KELRSSNVFDLEEIMREFNSYKQRVKYVEDKV.

In some of the same embodiments and some other embodiments, the portion of the native intravirion domain comprises an N-terminal or C-terminal truncation from the full length native intravirion domain amino acid sequence. In some preferred embodiments, the truncation is at the N-terminus of the native intravirion domain amino acid sequence. By way of an illustrative example, the modified gB polypeptide may comprise an amino acid sequence corresponding to amino acid residues 6 to 130 of the full length native intravirion domain (i.e., the amino acid sequence set forth in SEQ ID NO: 4). An exemplary sequence of suitable portion of the intravirion domain is identified in SEQ ID NO: 6, and set forth below.

[SEQ ID NO: 6]
LCTQPLQNLFPYLVSADGTTVTSGSTKDTSLQAPPSYEESVYNSGRKGP
GPPSSDASTAAPPYTNEQAYQMLLALARLDAEQRAQQNGTDSLDGQTGT
QDKGQKPNLLDRLRHRKNGYRHLKDSDEEENV.

By way of an illustrative example, a suitable modified gB polypeptide amino acid sequence is identified in SEQ ID NO: 4, and set forth below.

[SEQ ID NO: 4]
MEFGLSWLFLVAILKGVQCSSSTSHATSSTHNGSHTSRTTSAQTRSVYS
QHVTSSEAVSHRANETIYNTTLKYGDVVGVNTTKYPYRVCSMAQGTDLI
RFERNIICTSMKPINEDLDEGIMVVYKRNIVAHTFKVRVYQKVLTFRRS
YAYIYTTYLLGSNTEYVAPPMWEIHHINKFAQCYSSYSRVIGGTVFVAY
HRDSYENKTMQLIPDDYSNTHSTRYVTVKDQWHSRGSTWLYRETCNLNC
MLTITTARSKYPYHFFATSTGDVVYISPFYNGTNRNASYFGENADKFFI
FPNYTIVSDFGRPNAAPETHRLVAFLERADSVISWDIQDEKNVTCQLTF
WEASERTIRSEAEDSYHFSSAKMTATFLSKKQEVNMSDSALDCVRDEAI
NKLQQIFNTSYNQTYEKYGNVSVFETSGGLVVFWQGIKQKSLVELERLA
NRSSLNITHQTTQSTSDNNTTHLSSMESVHNLVYAQLQFTYDTLRGYIN
RALAQIAEAWCVDQRRTLEVFKELSKINPSAILSAIYNKPIAARFMGDV
LGLASCVTINQTSVKVLRDMNVKESPGRCYSRPVVIFNFANSSYVQYGQ
LGEDNEILLGNHRTEECQLPSLKIFIAGNSAYEYVDYLFKRMIDLSSIS
TVDSMIALDIDPLENTDFRVLELYSQKELRSSNVFDLEEIMREFNSYKQ
RVKYVEDKVLCTQPLQNLFPYLVSADGTTVTSGSTKDTSLQAPPSYEES
VYNSGRKGPGPPSSDASTAAPPYTNEQAYQMLLALARLDAEQRAQQNGT
DSLDGQTGTQDKGQKPNLLDRLRHRKNGYRHLKDSDEEENV.

Variant proteins encompassed by the present invention are biologically active, that is, they continue to possess a desired biological activity of the native protein (e.g., eliciting an immune response to CMV). Such variants may result from, for example, genetic polymorphism or from human manipulation.

A gB polypeptide may be altered in various ways including amino acid substitutions, deletions, truncations, and insertions. Methods for such manipulations are generally known in the art. For example, amino acid sequence variants of gB peptides or polypeptides can be prepared by mutations in the DNA. Methods for mutagenesis and nucleotide sequence alterations are well known in the art (see, for example, Kunkel (1985, Proc. Natl. Acad. Sci. USA. 82:488-492), Kunkel et al., (1987, Methods in Enzymol, 154:367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of gB peptides or polypeptides. Recursive ensemble mutagenesis (REM), a technique which enhances the frequency of functional mutants in the libraries, can be used in combination with the screening assays to identify gB variants (see, Arkin and Yourvan (1992) Proc. Natl. Acad. Sci. USA 89:7811-7815; Delgrave et al., (1993) Protein Engineering, 6:327-331). Conservative substitutions, such as exchanging one amino acid with another having similar properties, may be desirable as discussed in more detail below.

Variant gB polypeptides may contain conservative amino acid substitutions at various locations along their sequence, as compared to a parent (e.g., naturally occurring or reference) gB protein amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington DC; and by Gonnet et al., (1992, Science, 256 (5062): 14430-1445), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally occurring protein amino acids, sub-classification according to this scheme is presented in Table 3.

TABLE 3
AMINO ACIDS SUB-CLASSIFICATION

| Sub-Classes | Amino Acids |
|---|---|
| Acidic | Aspartic acid, Glutamic acid |
| Basic | Noncyclic: Arginine, lysine; |
| | Cyclic: histidine |
| Charged | Aspartic acid, glutamic acid, arginine, |
| | lysine, histidine |
| Small | Glycine, serine, alanine, threonine, proline |
| Nonpolar/neutral | Alanine, glycine, isoleucine, leucine, |
| | methionine, phenylalanine, proline, |
| | tryptophan, valine |
| Polar/neutral | Asparagine, histidine, glutamine, cysteine, |
| | serine, threonine, tyrosine |
| Polar/negative | Aspartic acid, glutamic acid |
| Polar/positive | Lysine, arginine |
| Polar/large | Asparagine, glutamine |
| Polar | Arginine, asparagine, aspartic acid, cysteine, |
| | glutamic acid, glutamine, histidine, lysine, |
| | serine, threonine, tyrosine |
| Hydrophobic | Tyrosine, valine, isoleucine, leucine, |
| | methionine, phenylalanine, tryptophan |
| Aromatic | Tryptophan, tyrosine, phenylalanine |
| Residues that influence | Glycine and proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have. A major effect on the properties of the resulting variant polypeptide. Whether an amino acid change results in a functional gB polypeptide can readily be determined by assaying its activity. Conservative substitutions are shown in Table 4 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 4
EXEMPLARY AND PREFERRED AMINO
ACID SUBSTITUTIONS

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | See |
| Gln | Asn, His, Lys | Asn |
| Glu | Asp, Lys | Asp |
| Gly | Pro | Pro |
| His | Asn, Gln, Lys, Arg | Arg |
| Ile | Leu, Val, Met, Ala, | Leu |
| | Phe, Norleu | |
| Leu | Norleu, Ile, Val, Met, | Ile |
| | Ala, Phe | |
| Lys | Arg, Gln, Asn | Arg |
| Met | Leu, Ile, Phe | Leu |
| Phe | Leu, Vel, Ile, Ala | Leu |
| Pro | Gly | Gly |
| Ser | Thr | Thr |
| Thr | Ser | Ser |
| Trp | Tyr | Tyr |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm: C. Brown Publishers (1993).

Thus, a predicted non-essential amino acid residue in a gB polypeptide is typically replaced with another amino acid residue from the same side chain family. Alternatively, mutations can be introduced randomly along all or part of an gB protein gene coding sequence, such as by saturation mutagenesis, and the resultant mutants can be screened for an activity of the parent polypeptide, as described for example herein, to identify mutants which retain that activity. Following mutagenesis of the coding sequences, the encoded polypeptide can be expressed recombinantly and its activity determined. A “nonessential” amino acid residue is a residue that can be altered from the wild-type sequence of an embodiment peptide or polypeptide without abolishing or substantially altering one or more of its activities. Suitably, the alteration does not substantially alter one of these activities, for example, the activity is at least 20%, 40%, 60%, 70% or 80% of wild-type. By contrast, an “essential” amino acid residue is a residue that, when altered from the wild-type sequence of a reference gB polypeptide, results in abolition of an activity of the parent molecule such that less than 20% of the wild-type activity is present. For example, such essential amino acid residues include those that are conserved in gB proteins across different species.

Accordingly, the present invention also contemplates as gB polypeptides, variants of the naturally occurring gB polypeptide sequences or their biologically active fragments, wherein the variants are distinguished from the naturally-occurring sequence by the addition, deletion, or substitution of one or more amino acid residues. In general, variants will display at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% similarity to a parent or reference gB protein sequence as, for example, set forth in any one of SEQ ID NOs: 1-3, as determined by sequence alignment programs described elsewhere herein using default parameters. Desirably, variants will have at least 40%, 45%, 50%, 51%, 52%, 53%, 54%, 55%, 56%, 57%, 58%, 59%, 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to a parent gB protein sequence as, for example, set forth in any one of SEQ ID NOS: 1-3, as determined by sequence alignment programs described elsewhere herein using default parameters. Variants of a wild-type gB protein, which fall within the scope of a variant polypeptide, may differ from the wild-type molecule generally by as much 15, 14, 13, 12, or 11 amino acid residues or suitably by as few as 10, 9, 8, 7, 6, 5, 4, 3, 2, or 1 amino acid residue(s). In some embodiments, a variant polypeptide differs from the corresponding sequences in any one of SEQ ID NOs: 1-3 by at least 1 but by less than or equal to 15, 14, 13, 12, 11, 10, 9, 8, 7, 6, 5, 4, 3, or 2 amino acid residues. In other embodiments, it differs from the corresponding sequence in any one of SEQ ID NO: 1 by at least one 1% but less than or equal to 20%, 19%, 18%, 17%, 16%, 15%, 14%, 13%, 12%, 11%, 10%, 9%, 8%, 7%, 6%, 5%, 4%, 3%, or 2% of the residues. If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution, as discussed in more detail below.

The modified gB polypeptides of the present invention also encompass gB polypeptides comprising amino acids with modified side chains, incorporation of unnatural amino acid residues and/or their derivatives during peptide, polypeptide or protein synthesis and the use of cross-linkers and other methods which impose conformational constraints on the peptides, portions and variants of the invention. Examples of side chain modifications include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBRₜ; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivatisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulfides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in Table 5.

TABLE 5
NON-CONVENTIONAL AMINO ACIDS

| | |
|---|---|
| α-aminobutyric acid | L-N-methylalanine |
| α-amino-α-methylbutyrate | L-N-methylarginine |
| Aminocyclopropane-carboxylate | L-N-methylasparagine |
| Aminoisobutyric acid | L-N-methylaspartic acid |
| Aminonorbornyl-carboxylate | L-N-methylcysteine |
| Cyclohexylalanine | L-N-methylglutamine |
| Ayclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-γ-aminobutyrate |
| D-α-methylalanine | α-methylcyclohexylalanine |
| D-α-methylasparagine | D-α-methyl-α-napthylalanine |
| D-α-methylaspartate | D-α-methylpenicillamine |
| D-α-methylcysteine | N-(4-aminobutyl)glycine |
| D-α-methylhistidine | N-(2-aminoethyl)glycine |
| D-α-methylisoleucine | N-(3-aminopropyl)glycine |
| D-α-methylleucine | N-amino-α-methylbutyrate |
| D-α-methyllysine | α-napthylalanine |
| D-α-methylmethionine | N-benzylglycine |
| D-α-methylornithine | N-(2-carbamylediyl)glycine |
| D-α-methylpheylalanine | N-(caramylmethyl)glycine |
| D-α-methylproline | N-(2-carboxyethyl)glycine |
| D-α-methylserine | N-(carboxymethyl)glycine |
| D-α-methylthreonine | N-cyclobutylglycine |
| D-α-methyltryptophan | N-cycloheptylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-2,2-diphenylethyl | N-(N-(3,3-diphenylpropyl |
| carbamylmethyl)glycine | carbnamylmethyl)glycine |
| 1-carboxy-1-1(2,2-diphenyl-ethyl | |
| amino)cyclopropane | |

Variant proteins encompassed by the present invention are biologically (e.g., immunologically) active, that is, they continue to possess the desired biological activity of the native protein. Such variants may result from, for example, genetic polymorphism or from human manipulation.

### 2.3 Heterologous Signal Sequence

The modified gB polypeptides of the present invention are typically secreted when expressed in a host cell (e.g., a mammalian cell). As such, in some preferred embodiments the modified gB polypeptide comprises an N-terminal heterologous signal peptide. Many signal peptides which facilitate secretion of the operatively linked peptide from a host cell are known in the art, and any such signal sequences may be suitable for use with the present invention. In some embodiments, the signal peptide directs translocation of the operatively linked immunogenic modified gB polypeptide to the endoplasmic reticulum, the cell membrane, the proteasome, the lysosome, or directs the immunogenic portion of gB to specific cell types or cell subsets.

In some embodiments, the heterologous signal is selected from the group consisting of an immunoglobulin signal sequence, tissue plasminogen activator (tPA) signal sequence, erythropoietin (EPO) signal sequence, VP22 HSV1 signal sequence, parathyroid hormone-related protein (PTHrP) N-terminal ER signal, calreticulin (CRT), adenovirus E3 signal sequence, or a flavivirus signal sequence including structural proteins (e.g., capsid (C), envelope (E), or premembrane (prM) proteins).

In some embodiments, the signal sequence comprises, or consists of, a target sequence which targets the encoded product to a desired cell type or cellular subset and may facilitate secretion or localisation of the operatively linked portion of the modified gB polypeptide. In some embodiments, the target sequence targets the operatively linked portion of the modified gB polypeptide to an immune cell. In some embodiments, the target sequence targets the operatively linked portion of modified gB polypeptide to an antigen presenting cell. In some embodiments, the target sequence targets the encoded product to the proteasome of a host cell. In some embodiments, the target sequence targets the operatively linked portion of the modified gB polypeptide to the endosome or lysosome of a host cell.

In some embodiments, the heterologous signal peptide an immunoglobulin (Ig) signal peptide. Many Ig signal peptide sequences are known in the art. In some embodiments, the signal peptide is derived from an immunoglobulin isotype selected from any one of IgA, IgD, IgE, IgG, and IgM. In one example, the heterologous signal peptide as described herein may comprise 10, 11, 12, 13, 14, 15, 16, 17, 18, 19, 20, 21, 22, 23, 24, 24, 25, 26, 27, 28, 29, 30, 31, 32, 33, 34, 35, 36, 37, 38, 39, or 40 amino acids. By way of an illustrative example, the amino acid sequence of a preferred signal peptide is the 18 amino acid IgG heavy chain signal peptide set forth in SEQ ID NO: 7, and set forth below:
MEFGLSWLFLVAILKGVQC [SEQ ID NO: 7].

In another example, the amino acid sequence of a preferred signal peptide is the 19 amino acid IgG heavy chain signal peptide identified in SEQ ID NO: 8, and set forth below:
MEFGLSWVFLVALFRGVQC [SEQ ID NO: 8].

In some embodiments, the N-terminal signal peptide has at least 80%, 85%, 90%, 95%, 98%, 99% or 100% sequence identity to the sequence set forth in SEQ ID NO: 7.

Suitably, the compositions comprise above 80% homotrimers of modified gB polypeptide, above 85% homotrimers of modified gB polypeptide, above 90% homotrimers of modified gB polypeptide, above 95% homotrimers of modified gB polypeptide, above 97% homotrimers of modified gB polypeptide, or above 98% homotrimers of modified gB polypeptide. In some embodiments, the preparation comprises above 99% homotrimers of modified gB polypeptide.

In some embodiments of this type, the compositions are substantially free of monomers of the modified gB polypeptide, and/or dimers of the modified gB polypeptide.

In some of the same embodiments and some other embodiments, the homotrimers of modified gB polypeptide dimerize to form hexameric complexes.

In some preferred embodiments, the gB polypeptide complex is in a prefusion isoform.

### 2.4 Nucleic Acid Compositions

The present invention also provides nucleic acid compositions that encode a modified gB protein as described above and/or elsewhere herein. In some embodiments, the isolated nucleic acid comprises, consists of, or consists essentially of the nucleotide sequence set forth in SEQ ID NO: 9 or a fragment, variant or derivative thereof.

Also contemplated are fragments and variants of the isolated nucleic acid.

The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence encoding the isolated protein of the invention (e.g., SEQ ID NO: 9).

Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence disclosed herein.

Fragments may comprise or consist of up to 20, 50, 100, 150, 200, 250, 300, 350, 400, 450, 500, 550, 600, 650, or 660 contiguous nucleotides present in any nucleotide sequence disclosed herein.

The present invention also contemplates nucleic acids that have been modified such as by taking advantage of codon sequence redundancy. In a more particular example, codon usage may be modified to optimise expression of a nucleic acid in a particular organism or cell type.

The invention further provides use of modified purines (for example, inosine, methylinosine and methyladenosine) and modified pyrimidines (for example, thiouridine and methylcytosine) in isolated nucleic acids of the invention.

It will be well appreciated by a person of skill in the art that the isolated nucleic acids of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In yet another embodiment, complementary nucleic acids hybridise to nucleic acids of the invention under high stringency conditions.

“Hybridise” and “hybridisation” are used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

“Stringency” as used herein, refers to temperature and ionic strength conditions, and presence or absence of certain organic solvents and/or detergents during hybridisation. The higher the stringency, the higher will be the required level of complementarity between hybridising nucleotide sequences.

“Stringent conditions” designates those conditions under which only nucleic acid having a high frequency of complementary bases will hybridise.

Stringent conditions are well-known in the art, such as described in Chapters 2.9 and 2.10 of Ausubel et al., supra, which are herein incorporated by reference. A skilled addressee will also recognise that various factors can be manipulated to optimise the specificity of the hybridisation. Optimisation of the stringency of the final washes can serve to ensure a high degree of hybridisation.

Complementary nucleotide sequences may be identified by blotting techniques that include a step whereby nucleotides are immobilised on a matrix (preferably a synthetic membrane such as nitrocellulose), a hybridisation step, and a detection step, typically using a labelled probe or other complementary nucleic acid. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridising the membrane bound DNA to a complementary nucleotide sequence. An alternative blotting step is used when identifying complementary nucleic acids in a cDNA or genomic DNA library, such as through the process of plaque or colony hybridization. Other typical examples of this procedure are described in Chapters 8-12 of Sambrook et al., MOLECULAR CLONING. A Laboratory Manual (Cold Spring Harbor Press, 1989).

Methods for detecting labelled nucleic acids hybridised to an immobilised nucleic acid are well known to practitioners in the art. Such methods include autoradiography, chemiluminescent, fluorescent and colorimetric detection.

Nucleic acids may also be isolated, detected and/or subjected to recombinant DNA technology using nucleic acid sequence amplification techniques.

Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-B replicase amplification, recombinase polymerase amplification (RPA) and helicase-dependent amplification, although without limitation thereto.

As used herein, an “amplification product” refers to a nucleic acid product generated by nucleic acid amplification.

Nucleic acid amplification techniques may include particular quantitative and semi-quantitative techniques such as qPCR, real-time PCR and competitive PCR, as are well known in the art.

In still a further aspect, the invention provides a genetic construct comprising the isolated nucleic acid of the previous aspect.

In particular embodiments, the genetic construct comprises the isolated nucleic acid operably linked or connected to one or more other genetic components. A genetic construct may be suitable for therapeutic delivery of the isolated nucleic acid or for recombinant production of the isolated protein of the invention in a host cell.

Broadly, the genetic construct can be in the form of, or comprises genetic components of, a plasmid, bacteriophage, a cosmid, a yeast or bacterial artificial chromosome as are well understood in the art. Genetic constructs may be suitable for maintenance and propagation of the isolated nucleic acid in bacteria or other host cells, for manipulation by recombinant DNA technology and/or expression of the nucleic acid or an encoded protein of the invention.

For the purposes of host cell expression, the genetic construct is an expression construct. Suitably, the expression construct comprises the nucleic acid of the invention operably linked to one or more additional sequences in an expression vector. An “expression vector” may be either a self-replicating extra-chromosomal vector such as a plasmid, or a vector that integrates into a host genome.

The term “operably connected” or “operably linked” as used herein means placing a structural gene under the regulatory control of a regulatory polynucleotide such as a promoter, which controls the transcription and optionally translation of the gene. For example, in the construction of heterologous promoter/structural gene combinations, it is generally preferred to position the genetic sequence or promoter at a distance from the gene transcription start site that is approximately the same as the distance between that genetic sequence or promoter and the gene it controls in its natural setting (i.e., the gene from which the genetic sequence or promoter is derived). As is known in the art, some variation in this distance can be accommodated without loss of function. Similarly, the preferred positioning of a regulatory sequence element with respect to a heterologous gene to be placed under its control is defined by the positioning of the element in its natural setting (i.e., the genes from which it is derived).

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, polyadenylation sequences, transcriptional start and termination sequences, translational start and termination sequences, and enhancer or activator sequences.

Constitutive, repressible or inducible promoters as known in the art are contemplated by the invention.

The expression construct may also include an additional nucleotide sequence encoding a fusion partner (typically provided by the expression vector) so that the recombinant protein is expressed as a fusion protein.

The expression construct may also include an additional nucleotide sequence encoding a selection marker such as ampR, neoR or kanR, although without limitation thereto.

In particular embodiments, the expression construct may be in the form of plasmid DNA, suitably comprising a promoter operable in an animal cell (e.g. a CMV, an A-crystallin or SV40 promoter). In other embodiments, the nucleic acid may be in the form of a viral construct such as an adenoviral, vaccinia, lentiviral or adeno-associated viral vector.

In another aspect, the invention relates to a host cell transformed with a nucleic acid molecule or a genetic construct described herein.

Suitable host cells for expression may be prokaryotic or eukaryotic. For example, suitable host cells may include but are not limited to mammalian cells (e.g. CHO, HeLa, Cos, NIH-3T3, HEK293T, Jurkat cells), yeast cells (e.g. Saccharomyces cerevisiae), insect cells (e.g. Sf9, Trichoplusia ni) utilised with or without a baculovirus expression system, plant cells (e.g. Chlamydomonas reinhardtii, Phaeodactylum tricornutum) or bacterial cells, such as E. coli. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2015), in particular Chapters 9 and 16.

Related aspects of the invention provide a method of producing the isolated protein described herein, including the steps of; (i) culturing the host cell of the previous aspect; and (ii) isolating said isolated protein from said host cell cultured in step (i).

In this regard, the recombinant protein may be conveniently prepared by a person skilled in the art using standard protocols, such as those hereinbefore provided.

### 3. Pharmaceutical Compositions

The modified gB polypeptides of the present invention can be used as active ingredients for the therapeutic treatment and/or prophylaxis of CMV infection. These therapeutic treatments and/or prophylactic agents can be administered to a subject either in isolation or as compositions where they are mixed with pharmaceutically acceptable carriers, diluents, and/or adjuvants.

Depending on the specific conditions being treated compositions for therapy and/or prophylaxis may be formulated and administered systemically or locally. Techniques for formulation and administration may be found in “Remington's Pharmaceutical Sciences,” Mack Publishing Co., Easton, Pa., latest edition. Suitable routes may, for example, include intradermal injection. For injection, the therapeutic agents of the invention may be formulated in aqueous solutions, preferably in physiologically compatible buffers such as Flanks' solution, Ringer's solution, or physiological saline buffer. For transmucosal administration, penetrants appropriate to the barrier to be permeated are used in the formulation. Such penetrants are generally known in the art. Intramuscular and subcutaneous injection is appropriate, for example, for administration of immunogenic compositions, vaccines and DNA vaccines. In some specific embodiments, the pharmaceutical compositions are formulated for intradermal administration.

The pharmaceutical compositions of the invention can be formulated readily using pharmaceutically acceptable carriers well known in the art into dosages suitable for administration. Such carriers enable the compounds of the invention to be formulated in dosage forms such as tablets, pills, capsules, liquids, gels, syrups, slurries, suspensions and the like, for administration to the subject to be treated. For example, a pharmaceutical composition formulated for oral ingestion will contain a suitable carrier, for example, selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to elicit a beneficial response in the patient over time, such as a reduction in the symptoms associated with the condition. The quantity of the therapeutic/prophylactic agent(s) to be administered may depend on the subject to be treated inclusive of the age, sex, weight and general health condition thereof. In this regard, precise amounts of the therapeutic/prophylactic agent(s) for administration will depend on the judgement of the practitioner. In determining the effective amount of the agent to be administered in the treatment or prophylaxis of the condition, the physician may evaluate tissue levels of a polypeptide antigen, and progression of the disease or condition. In any event, those of skill in the art may readily determine suitable dosages of the therapeutic and/or prophylactic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

From the foregoing, it will be appreciated that the agents of the invention may be used as therapeutic or prophylactic immunostimulating compositions and/or vaccines. Accordingly, the invention extends to the production of immunostimulating compositions containing as active compounds one or more of the therapeutic/prophylactic agents of the invention. Any suitable procedure is contemplated for producing such vaccines. Exemplary procedures include, for example, those described in NEW GENERATION VACCINES (1997, Levine et al., Marcel Dekker, Inc. New York, Basel Hong Kong).

Suitably, antigen-presenting cells contacted ex vivo with the modified gB polypeptide of the invention, as well as antigen-specific T lymphocytes generated with these antigen-presenting cells can be used as active compounds in immunostimulating compositions for prophylactic or therapeutic applications. The primed cells, which are preferably mature dendritic cells, can be injected with the modified polypeptide by any method that elicits an immune response into a syngeneic subject (i.e., a human). Preferably, antigen-presenting cells are injected back into the same subject from whom the source tissue/cells were obtained. The injection site may be subcutaneous, intraperitoneal, intramuscular, intradermal, or intravenous. The number of antigen-primed antigen-presenting cells reinjected back into the subject in need of treatment may vary depending on inter alia, the antigen and size of the individual. This number may range for example between about 10⁴ and 10⁸, and more preferably between about 10⁶ and 10⁷ antigen-primed antigen-presenting cells (e.g., dendritic cells). The antigen-presenting cells should be administered in a pharmaceutically acceptable carrier, which is non-toxic to the cells and the individual. Such carrier may be the growth medium in which the antigen-presenting cells were grown, or any suitable buffering medium such as phosphate buffered saline.

In one embodiment, the antigen-primed antigen-presenting cells of the invention could also be used for generating large numbers of CD4⁺ CTL, for adoptive transfer to immunosuppressed individuals who are unable to mount normal immune responses. For example, antigen specific CD4⁺ cytotoxic T lymphocyte (CTL) can be adoptively transferred for therapeutic purposes in subjects afflicted with CMV infection.

The effectiveness of the immunisation may be assessed using any suitable technique. For example, CTL lysis assays may be employed using stimulated splenocytes or peripheral blood mononuclear cells (PBMC) on peptide coated or recombinant virus infected cells using ⁵¹Cr labelled target cells. Such assays can be performed using for example any mammalian cells (Allen et al., 2000, J. Immunol. 164 (9): 4968-4978; also Woodberry et al., infra). Alternatively, the efficacy of the immunisation may be monitored using one or more techniques including, but not limited to, HLA class I tetramer staining-of both fresh and stimulated PBMCs (see for example Allen et al., supra), proliferation assays (Allen et al., supra), Elispot assays and intracellular cytokine staining (Allen et al., supra), ELISA assays for detecting linear B cell responses; and Western blots of cell sample expressing the synthetic polynucleotides. Particularly relevant will be the cytokine profile of T-cells activated by antigen, and more particularly the production and secretion of IFN-γ, IL-2, IL-4, IL-5, IL-10, TGF-β and TNF.

The compositions of the present invention are suitably pharmaceutical compositions. The pharmaceutical compositions often comprise one or more “pharmaceutically acceptable carriers.” These include any carrier which does not itself induce the production of antibodies harmful to the individual receiving the composition. Suitable carriers typically are large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. A composition may also contain a diluent, such as water, saline, glycerol, etc. Additionally, an auxiliary substance, such as a wetting or emulsifying agent, pH buffering substance, and the like, may be present. A thorough discussion of pharmaceutically acceptable components is available in Gennaro (2000) Remington: The Science and Practice of Pharmacy. 20th ed, ISBN: 0683306472.

The pharmaceutical compositions may include various salts, excipients, delivery vehicles and/or auxiliary agents as are disclosed, e.g., in U.S. patent application Publication No. 2002/0019358, published Feb. 14, 2002.

The immunostimulating compositions according to the present invention can contain a physiologically acceptable diluent or excipient such as water, phosphate buffered saline and saline. They may also include an adjuvant as is well known in the art. Suitable adjuvants include, but are not limited to: oligonucleotide adjuvants, surface active substances such as hexadecylamine, octadecylamine, octadecyl amino acid esters, lysolecithin, dimethyldioctadecylammonium bromide, N, N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, aluminium hydroxide or alum; lymphokines, QuilA and immune stimulating complexes (ISCOMS).

The adjuvant in the composition suitably comprises one or more TLR agonist.

The term “TLR agonist”, as used herein, refers to a molecule that is capable of causing a signalling response through a TLR signalling pathway, either as a ligand directly or indirectly through of the generation of endogenous or exogenous ligand. The agonist ligands of the TLR receptors can be natural ligands of the TLR receptor, or functionally equivalent variants thereof that retain the ability to bind to the TLR receptor and induce costimulation signals therein. The TLR agonist may also be an agonist antibody against the TLR receptor, or a functionally equivalent variant thereof, that is capable of specifically binding to the TLR receptor and, more particularly, to the extracellular domain of said receptor, and inducing some of the immune signals controlled by this receptor and associated proteins. The specificity of binding can be for the human TLR receptor or for a human homologous TLR receptor of a different species.

In particular embodiments, the one or more TLR agonists include a TLR4 agonist and/or a TLR9 agonist. More particularly, the TLR agonist is or comprises a TLR9 agonist.

Exemplary TLR4 agonists are lipolopysacchardides (LPS) or derivatives or components of LPS. These include Monophosphoryl lipid A (MPL®) derived from Salmonella minnesota and synthetic TLR4 agonists such as aminoalkyl glucosaminide phosphates (AGPs) and Phosphorylated HexaAcyl Disaccharide (PHAD) and derivatives thereof (e.g., 3D-PHAD, 3D (6-acyl)-PHAD). A preferred TLR4 agonist is MPL.

TLR9 recognises specific unmethylated CpG oligonucleotides (ODN) sequences that distinguish microbial DNA from mammalian DNA. CpG ODNs oligonucleotides contain unmethylated CpG dinucleotides in particular sequence contexts (CpG motifs). These CpG motifs are present at a 20-fold greater frequency in bacterial DNA compared to mammalian DNA. Three types of stimulatory ODNs have been described: type A, B and C.

Non-limiting examples of TLR9 agonists include CpG ODN1018, CpG ODN2006, CpG ODN2216, CpG ODN1826 and CpG ODN2336, although without limitation thereto. In some embodiments, the TLR9 agonist is or comprises CpG ODN1018 and/or CpG ODN2006. In one preferred embodiment, the TLR9 agonist is CpG ODN1018. Also envisaged are amphiphile vaccine adjuvants, for example, amphiphile CpG adjuvants (e.g., Amphiphile CpG1018.

In particular embodiments, the TLR agonist is not MPL, CpG ODN1826, CpG ODN2006, CpG ODN2216 and/or CpG ODN2336.

Suitably, the pharmaceutical composition further comprises a pharmaceutically-acceptable carrier, diluent or excipient.

Certain compositions of the present invention can further include one or more adjuvants before, after, or concurrently with the polynucleotide. The term “adjuvant” refers to any material having the ability to (1) alter or increase the immune response to a particular antigen or (2) increase or aid an effect of a pharmacological agent. It should be noted, with respect to polynucleotide vaccines, that an “adjuvant,” can be a transfection facilitating material. Similarly, certain “transfection facilitating materials” described supra, may also be an “adjuvant”. An adjuvant maybe used with a composition comprising a polynucleotide of the present invention. In a prime-boost regimen, as described herein, an adjuvant may be used with either the priming immunisation, the booster immunisation, or both. Suitable adjuvants include, but are not limited to, cytokines and growth factors; bacterial components (e.g., endotoxins, in particular superantigens, exotoxins and cell wall components); aluminium-based salts; calcium-based salts; silica; polynucleotides; toxoids; serum proteins, viruses and virally-derived materials, poisons, venoms, imidazoquiniline compounds, poloxamers, and cationic lipids.

A great variety of materials have been shown to have adjuvant activity through a variety of mechanisms. Any compound which may increase the expression, antigenicity or immunogenicity of the polypeptide is a potential adjuvant. The present invention provides an assay to screen for improved immune responses to potential adjuvants. Potential adjuvants which may be screened for their ability to enhance the immune response according to the present invention include, but are not limited to: Montanide, inert carriers, such as alum, bentonite, latex, and acrylic particles; PLURONIC block polymers, such as TITERMAX (block copolymer CRL-8941, squalene (a metabolizable oil) and a microparticulate silica stabilizer); depot formers, such as Freund's adjuvant, surface active materials, such as saponin, lysolecithin, retinal, Quil A, liposomes, and PLURONIC polymer formulations; macrophage stimulators, such as bacterial lipopolysaccharide; alternate pathway complement activators, such as insulin, zymosan, endotoxin, and levamisole; and non-ionic surfactants, such as poloxamers, poly(oxyethylene)-poly(oxypropylene) tri-block copolymers. Also included as adjuvants are transfection-facilitating materials, such as those described above.

The Montanide adjuvants are based on purified squalene and squalene, emulsified with highly purified mannide mono-oleate. There are several types of Montanide, including ISA 50V, 51, 206, and 720. ISA 50V, 51 and 720 are water-in-oil (W/O) emulsions, which ISA 206 is a W/O-in-water emulsion. Emulsions of Montanide ISA51 and 720 are composed of metabolisable squalene-based oil with a mannide mono-oleate emulsifier.

Poloxamers which may be screened for their ability to enhance the immune response according to the present invention include, but are not limited to, commercially available poloxamers such as PLURONIC surfactants, which are block copolymers of propylene oxide and ethylene oxide in which the propylene oxide block is sandwiched between two ethylene oxide blocks. Examples of PLURONIC surfactants include PLURONIC L121 poloxamer (ave. MW: 4400; approx. MW of hydrophobe, 3600; approx wt % of hydrophile, 10%), PLURONIC L101 poloxamer (ave. MW: 3800; approx. MW of hydrophobe, 3000; approx wt. % of hydrophile, 10%), PLURONIC L81 poloxamer (ave. MW: 2750; approx. MW of hydrophobe, 2400; approx wt. % of hydrophile, 10%), PLURONIC L61 poloxamer (ave. MW: 2000; approx. MW of hydrophobe, 1800; approx wt. % of hydrophile, 10%), PLURONIC L31 poloxamer (ave. MW: 1 100; approx. MW of hydrophobe, 900; approx wt. % of hydrophile, 10%), PLURONIC L122 poloxamer (ave. MW: 5000; approx. MW of hydrophobe, 3600; approx wt. % of hydrophile, 20%), PLURONIC L92 poloxamer (ave. MW: 3650; approx. MW of hydrophobe, 2700; approx wt. % of hydrophile, 20%), PLURONIC L72 poloxamer (ave. MW: 2750; approx. MW of hydrophobe, 2100; approx wt. % of hydrophile, 20%), PLURONIC L62 poloxamer (ave. MW: 2500; approx. MW of hydrophobe, 1800; approx wt. % of hydrophile, 20%), PLURONIC L42 poloxamer (ave. MW: 1630; approx. MW of hydrophobe, 1200; approx wt. % of hydrophile, 20%),

PLURONIC L63 poloxamer (ave. MW: 2650; approx. MW of hydrophobe, 1800; approx wt. % of hydrophile, 30%), PLURONIC L43 poloxamer (ave. MW: 1850; approx. MW of hydrophobe, 1200; approx wt. % of hydrophile, 30%), PLURONIC L64 poloxamer (ave. MW: 2900; approx. MW of hydrophobe, 1800; approx wt. % of hydrophile, 40%), PLURONIC L44 poloxamer (ave. MW: 2200; approx. MW of hydrophobe, 1200; approx wt. % of hydrophile, 40%), PLURONIC L35 poloxamer (ave. MW: 1900; approx. MW of hydrophobe, 900; approx wt. % of hydrophile, 50%), PLURONIC P123 poloxamer (ave. MW: 5750; approx. MW of hydrophobe, 3600; approx wt. % of hydrophile, 30%), PLURONIC P103 poloxamer (ave. MW: 4950; approx. MW of hydrophobe, 3000; approx wt. % of hydrophile, 30%), PLURONIC P104 poloxamer (ave. MW: 5900; approx. MW of hydrophobe, 3000; approx wt. % of hydrophile, 40%), PLURONIC P84 poloxamer (ave. MW: 4200; approx. MW of hydrophobe, 2400; approx wt. % of hydrophile, 40%), PLURONIC P105 poloxamer (ave. MW: 6500; approx. MW of hydrophobe, 3000; approx wt. % of hydrophile, 50%), PLURONIC P85 poloxamer (ave. MW: 4600; approx. MW of hydrophobe, 2400; approx wt. % of hydrophile, 50%), PLURONIC P75 poloxamer (ave. MW: 4150; approx. MW of hydrophobe, 2100; approx wt. % of hydrophile, 50%), PLURONIC P65 poloxamer (ave. MW: 3400; approx. MW of hydrophobe, 1800; approx wt. % of hydrophile, 50%), PLURONIC F127 poloxamer (ave. MW: 12600; approx. MW of hydrophobe, 3600; approx wt. % of hydrophile, 70%), PLURONIC F98 poloxamer (ave. MW: 13000; approx. MW of hydrophobe, 2700; approx wt. % of hydrophile, 80%), PLURONIC F87 poloxamer (ave. MW: 7700; approx. MW of hydrophobe, 2400; approx wt. % of hydrophile, 70%), PLURONIC F77 poloxamer (ave. MW: 6600; approx. MW of hydrophobe, 2100; approx wt. % of hydrophile, 70%), PLURONIC F108 poloxamer (ave. MW: 14600; approx. MW of hydrophobe, 3000; approx wt. % of hydrophile, 80%), PLURONIC F98 poloxamer (ave. MW: 13000; approx. MW of hydrophobe, 2700; approx wt. % of hydrophile, 80%), PLURONIC F88 poloxamer (ave. MW: 1 1400; approx. MW of hydrophobe, 2400; approx wt. % of hydrophile, 80%), PLURONIC F68 poloxamer (ave. MW: 8400; approx. MW of hydrophobe, 1800; approx wt. % of hydrophile, 80%), PLURONIC F38 poloxamer (ave. MW: 4700; approx. MW of hydrophobe, 900; approx wt. % of hydrophile, 80%).

Reverse poloxamers which may be screened for their ability to enhance the immune response according to the present invention include, but are not limited to PLURONIC R 31 R1 reverse poloxamer (ave. MW: 3250; approx. MW of hydrophobe, 3100; approx wt. % of hydrophile, 10%), PLURONIC R25R1 reverse poloxamer (ave. MW: 2700; approx. MW of hydrophobe, 2500; approx wt. % of hydrophile, 10%), PLURONIC R 17R1 reverse poloxamer (ave. MW: 1900; approx. MW of hydrophobe, 1700; approx wt. % of hydrophile, 10%), PLURONIC R 31 R2 reverse poloxamer (ave. MW: 3300; approx. MW of hydrophobe, 3100; approx wt. % of hydrophile, 20%), PLURONIC R 25R2 reverse poloxamer (ave. MW: 3100; approx. MW of hydrophobe, 2500; approx wt. % of hydrophile, 20%), PLURONIC R 17R2 reverse poloxamer (ave. MW: 2150; approx. MW of hydrophobe, 1700; approx wt. % of hydrophile, 20%), PLURONIC R 12R3 reverse poloxamer (ave. MW: 1800; approx. MW of hydrophobe, 1200; approx wt. % of hydrophile, 30%), PLURONIC R 31 R4 reverse poloxamer (ave. MW: 4150; approx. MW of hydrophobe, 3100; approx wt. % of hydrophile, 40%), PLURONIC R 25R4 reverse poloxamer (ave. MW: 3600; approx. MW of hydrophobe, 2500; approx wt. % of hydrophile, 40%), PLURONIC R 22R4 reverse poloxamer (ave. MW: 3350; approx. MW of hydrophobe, 2200; approx wt. % of hydrophile, 40%), PLURONIC R17R4 reverse poloxamer (ave. MW: 3650; approx. MW of hydrophobe, 1700; approx wt. % of hydrophile, 40%), PLURONIC R 25R5 reverse poloxamer (ave. MW: 4320; approx. MW of hydrophobe, 2500; approx wt. % of hydrophile, 50%), PLURONIC R10R5 reverse poloxamer (ave. MW: 1950; approx. MW of hydrophobe, 1000; approx wt. % of hydrophile, 50%), PLURONIC R 25R8 reverse poloxamer (ave. MW: 8550; approx. MW of hydrophobe, 2500; approx wt. % of hydrophile, 80%), PLURONIC R 17R8 reverse poloxamer (ave. MW: 7000; approx. MW of hydrophobe, 1700; approx wt. % of hydrophile, 80%), and PLURONIC R 10R8 reverse poloxamer (ave. MW: 4550; approx. MW of hydrophobe, 1000; approx wt. % of hydrophile, 80%).

Other commercially available poloxamers which may be screened for their ability to enhance the immune response according to the present invention include compounds that are block copolymer of polyethylene and polypropylene glycol such as SYNPERONIC L121 (ave. MW: 4400), SYNPERONIC L122 (ave. MW: 5000), SYNPERONIC P104 (ave. MW: 5850), SYNPERONIC P105 (ave. MW: 6500), SYNPERONIC P123 (ave. MW: 5750), SYNPERONIC P85 (ave. MW: 4600) and SYNPERONIC P94 (ave. MW: 4600), in which L indicates that the surfactants are liquids, P that they are pastes, the first digit is a measure of the molecular weight of the polypropylene portion of the surfactant and the last digit of the number, multiplied by 10, gives the percent ethylene oxide content of the surfactant; and compounds that are nonylphenyl polyethylene glycol such as SYNPERONIC NP10 (nonylphenol ethoxylated surfactant-10% solution), SYNPERONIC NP30 (condensate of 1 mole of nonylphenol with 30 moles of ethylene oxide) and SYNPERONIC NP5 (condensate of 1 mole of nonylphenol with 5.5 moles of naphthalene oxide).

Other poloxamers which may be screened for their ability to enhance the immune response according to the present invention include: (a) a polyether block copolymer comprising an A-type segment and a B-type segment, wherein the A-type segment comprises a linear polymeric segment of relatively hydrophilic character, the repeating units of which contribute an average Hansch-Leo fragmental constant of about-0.4 or less and have molecular weight contributions between about 30 and about 500, wherein the B-type segment comprises a linear polymeric segment of relatively hydrophobic character, the repeating units of which contribute an average Hansch-Leo fragmental constant of about-0.4 or more and have molecular weight contributions between about 30 and about 500, wherein at least about 80% of the linkages joining the repeating units for each of the polymeric segments comprise an ether linkage; (b) a block copolymer having a polyether segment and a polycation segment, wherein the polyether segment comprises at least an A-type block, and the polycation segment comprises a plurality of cationic repeating units; and (c) a polyether-polycation copolymer comprising a polymer, a polyether segment and a polycationic segment comprising a plurality of cationic repeating units of formula-NH− RO, wherein RO is a straight chain aliphatic group of 2 to 6 carbon atoms, which may be substituted, wherein said polyether segments comprise at least one of an A-type of B-type segment. See U.S. Pat. No. 5,656,61 1. Other poloxamers of interest include CRL1005 (12 kDa, 5% POE), CRL8300 (11 kDa, 5% POE), CRL2690 (12 kDa, 10% POE), CRL4505 (15 kDa, 5% POE) and CRL1415 (9 kDa, 10% POE).

Other auxiliary agents which may be screened for their ability to enhance the immune response according to the present invention include, but are not limited to, Acacia (gum arabic); the poloxyethylene ether R—O—(C2H40)x-H (BRIJ), e.g., polyethylene glycol dodecyl ether (BRIJ 35, x=23), polyethylene glycol dodecyl ether (BRIJ 30, x=4), polyethylene glycol hexadecyl ether (BRIJ 52 x=2), polyethylene glycol hexadecyl ether (BRIJ 56, x=10), polyethylene glycol hexadecyl ether (BRIJ 58P, x=20), polyethylene glycol octadecyl ether (BRIJ 72, x=2), polyethylene glycol octadecyl ether (BRIJ 76, x=10), polyethylene glycol octadecyl ether (BRIJ® 78P, x=20), polyethylene glycol oleyl ether (BRIJ 92V, x=2), and polyoxyl 10 oleyl ether (BRIJ 97, x=10); poly-D-glucosamine (chitosan); chlorbutanol; cholesterol; diethanolamine; digitonin; dimethylsulfoxide (DMSO), ethylenediamine tetraacetic acid (EDTA); glyceryl monosterate; lanolin alcohols; mono- and di-glycerides; monoethanolamine; nonylphenol polyoxyethylene ether (NP-40); octylphenoxypolyethoxyethanol (NONIDET NP-40 from Amresco); ethyl phenol poly (ethylene glycol ether) n, n=| 1 (NONIDET P40 from Roche); octyl phenol ethylene oxide condensate with about 9 ethylene oxide units (NONIDET P40); IGEPAL CA 630 ((octyl phenoxy) polyethoxyethanol; structurally same as NONIDET NP-40); oleic acid; oleyl alcohol; polyethylene glycol 8000; polyoxyl 20 cetostearyl ether; polyoxyl 35 castor oil; polyoxyl 40 hydrogenated castor oil; polyoxyl 40 stearate; polyoxyethylene sorbitan monolaurate (polysorbate 20, or TWEEN-20; polyoxyethylene sorbitan monooleate (polysorbate 80, or TWEEN-80); propylene glycol diacetate; propylene glycol monstearate; protamine sulfate; proteolytic enzymes; sodium dodecyl sulfate (SDS); sodium monolaurate; sodium stearate; sorbitan derivatives (SPAN), e.g., sorbitan monopalmitate (SPAN 40), sorbitan monostearate (SPAN 60), sorbitan tristearate (SPAN 65), sorbitan monooleate (SPAN 80), and sorbitan trioleate (SPAN 85); 2,6,10,15,19,23-hexamethyl-2,6,10,14,18,22-tetracosa-hexaene (squalene); stachyose; stearic acid; sucrose; surfactin (lipopeptide antibiotic from Bacillus subtilis); dodecylpoly(ethyleneglycolether) 9 (THESIT) MW 582.9; octyl phenol ethylene oxide condensate with about 9-10 ethylene oxide units (TRITON X-100); octyl phenol ethylene oxide condensate with about 7-8 ethylene oxide units (TRITON X-1 14); tris(2-hydroxyethyl)amine (trolamine); and emulsifying wax.

In certain adjuvant compositions, the adjuvant is a cytokine. A composition of the present invention can comprise one or more cytokines, chemokines, or compounds that induce the production of cytokines and chemokines, or a polynucleotide encoding one or more cytokines, chemokines, or compounds that induce the production of cytokines and chemokines. Examples include, but are not limited to, granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFN-α), interferon beta (IFN-β), interferon gamma (IFN-γ), interferon omega (IFNω), interferon tau (IFNT), interferon gamma inducing factor I (IGIF), transforming growth factor beta (TGF-b), RANTES (regulated upon activation, normal T-cell expressed and presumably secreted), macrophage inflammatory proteins (e.g., MIP-1a and MIP-1B), Leishmania elongation initiating factor (LEIF), and Fit-3 ligand.

In certain compositions of the present invention, the polynucleotide construct may be complexed with an adjuvant composition comprising (±)-N-(3-aminopropyl)-N, N-dimethyl-2,3-bis(syn-9-tetradeceneyloxy)-1-propanaminium bromide (GAP-DMORIE). The composition may also comprise one or more co-lipids, e.g., 1,2-dioleoyl-sn-glycero-3-phosphoethanolamine (DOPE), 1,2-diphytanoyl-sn-glycero-3-phosphoethanolamine (DPyPE), and/or 1,2-dimyristoyl-glycer-3-phosphoethanolamine (DMPE). An adjuvant composition comprising GAP-DMORIE and DPyPE at a 1:1 molar ratio is referred to herein as VAXFECTIN adjuvant. See, e.g., PCT Publication No. WO 00/57917.

In other embodiments, the polynucleotide itself may function as an adjuvant as is the case when the polynucleotides of the invention are derived, in whole or in part, from bacterial DNA. Bacterial DNA containing motifs of unmethylated CpG-dinucleotides (CpG-DNA) triggers innate immune cells in vertebrates through a pattern recognition receptor (including toll receptors such as TLR 9) and thus possesses potent immunostimulatory effects on macrophages, dendritic cells and B-lymphocytes. See, e.g., Wagner, H., Curr. Opin. Microbiol. 5:62-69 (2002); Jung, J. et al., J. Immunol. 169:2368-73 (2002); see also Klinman, D. M. et al., Proc. Natl Acad. Sci. U.S.A. 93:2879-83 (1996). Methods of using unmethylated CpG-dinucleotides as adjuvants are described in, for example, U.S. Pat. Nos. 6,207,646, 6,406,705 and 6,429,199.

Other suitable immunostimulatory molecules and adjuvants include, but are not limited to: a further TLR agonist, lipopolysaccharide and derivatives thereof such as MPL, Freund's complete or incomplete adjuvant, squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, aluminium hydroxide or alum; interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol′ EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

With regard to subunit vaccines, an example of such a vaccine may be formulated with ISCOMs, such as described in International Publication WO97/45444.

An example of a vaccine in the form of a water-in-oil formulation includes Montanide ISA 720, such as described in International Publication WO97/45444.

Any suitable procedure is contemplated for producing vaccine compositions. Exemplary procedures include, for example, those described in New Generation Vaccines (1997, Levine et al., Marcel Dekker, Inc. New York, Basel, Hong Kong), which is incorporated herein by reference.

The ability of an adjuvant to increase the immune response to an antigen is typically manifested by a significant increase in immune-mediated protection. For example, an increase in humoral immunity is typically manifested by a significant increase in the titre of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Nucleic acid molecules and/or polynucleotides of the present invention, e.g., plasmid DNA, mRNA, linear DNA or oligonucleotides, may be solubilised in any of various buffers. Suitable buffers include, for example, phosphate buffered saline (PBS), normal saline, Tris buffer, and sodium phosphate (e.g., 150 mM sodium phosphate). Insoluble polynucleotides may be solubilised in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a subject (e.g., human).

Compositions of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995). Although the composition may be administered as an aqueous solution, it can also be formulated as an emulsion, gel, solution, suspension, lyophilised form, or any other form known in the art. In addition, the composition may contain pharmaceutically acceptable additives including, for example, diluents, binders, stabilisers, and preservatives.

### 4. Dosage

The present invention is generally concerned with therapeutic and prophylactic compositions. The compositions will comprise an “effective amount” of the compositions defined herein, such that an amount of the antigen can be produced in vivo so that an immune response is generated in the individual to which it is administered. The exact amount necessary will vary depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, an “effective amount” will fall in a relatively broad range that can be determined through routine trials.

Dosage amount and interval may be adjusted individually to provide plasma levels of the active compound(s) which are sufficient to maintain target antigen-reducing effects or effects that ameliorate the disease or condition. Usual patient dosages for systemic administration range from about 1 μg-500 μg, commonly from about 20 μg-200 μg, and typically from about 25 μg-150 μg.

Alternatively, one may administer the agent in a local rather than systemic manner, for example, via injection of the compound directly into a tissue, often in a depot or sustained release formulation. Furthermore, one may administer the agent in a targeted drug delivery system, for example, in a liposome coated with tissue-specific antibody. The liposomes will be targeted to and taken up selectively by the tissue.

For any compound used in the method of the invention, the effective dose can be estimated initially from cell culture assays. For example, a dose can be formulated in animal models to achieve a circulating concentration range that includes the IC50 as determined in cell culture (e.g., the concentration of a test agent, which achieves a half-maximal reduction in target antigen). Such information can be used to more accurately determine useful doses in a mammal.

Toxicity and therapeutic efficacy of the compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in the subject. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the subject's condition. (See for example Fingl et al., 1975, in “The Pharmacological Basis of Therapeutics”, Ch. 1 p1).

The compositions of the present invention can be suitably formulated for injection. The composition may be prepared in unit dosage form in ampules, or in multidose containers. The polynucleotides may be present in such forms as suspensions, solutions, or emulsions in oily or preferably aqueous vehicles. Alternatively, the polynucleotide salt may be in lyophilised form for reconstitution, at the time of delivery, with a suitable vehicle, such as sterile pyrogen-free water. Both liquid as well as lyophilised forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Non-ionic materials, such as sugars, are preferred for adjusting tonicity, and sucrose is particularly preferred. Any of these forms may further comprise suitable formulatory agents, such as starch or sugar, glycerol or saline. The compositions per unit dosage, whether liquid or solid, may contain from 0.1% to 99% of polynucleotide material.

The unit dosage ampules or multidose containers, in which the polynucleotides are packaged prior to use, may comprise a hermetically sealed container enclosing an amount of polynucleotide or solution containing a polynucleotide suitable for a pharmaceutically effective dose thereof, or multiples of an effective dose. The polynucleotide is packaged as a sterile formulation, and the hermetically sealed container is designed to preserve sterility of the formulation until use.

The dosage to be administered depends to a large extent on the condition and size of the subject being treated as well as the frequency of treatment and the route of administration. Regimens for continuing therapy, including dose and frequency may be guided by the initial response and clinical judgment. The parenteral route of injection into the interstitial space of tissues is preferred, although other parenteral routes, such as inhalation of an aerosol formulation, may be required in specific administration, as for example to the mucous membranes of the nose, throat, bronchial tissue or lungs.

### 5. Methods of Use

Also encapsulated by the present invention is a method for treatment and/or prophylaxis of CMV infection, comprising administering to a subject (e.g., a human) in need of such treatment an effective amount of a composition as broadly described above and elsewhere herein.

In one embodiment, the modified gB polypeptides of the invention can also be used for generating large numbers of CD4⁺ CTL. For example, antigen specific CD4⁺ CTL can be adoptively transferred for therapeutic purposes in a subject (e.g., human) afflicted with a CMV infection.

In accordance with the present invention, it is proposed that compositions that include the modified gB polypeptides described above and/or elsewhere herein, find utility in the treatment or prophylaxis of a CMV infection. The compositions of the present invention may be used therapeutically after CMV infection is diagnosed.

When the compositions described above and elsewhere herein are used in prophylactic methods against CMV infection, such methods are suitably prime-boost vaccinations against a gB-specific antibodies that induce long-lasting humoral, cell-mediated and mucosal immune responses against the gB polypeptide.

In some embodiments the compositions of the present invention are administered in multiple doses in a prime-boost regimen, with the goal of inducing long-lived potent immunity against a gB polypeptide. Such strategies use a second dose of the composition to bolster immunity elicited by the priming dose.

Some embodiments of the present invention are based on the realisation that an optimal strategy for eliciting therapeutic and protective immunity against a gB polypeptide involves the generation of both a cellular and a humoral immune response to the CMV virus. The invention thus provides a multi-component administration strategy in which a first dose of the composition of the present invention primes the immune system by eliciting or inducing a first immune response, and a second dose of the composition of the present invention is used to boost or elicit a second immune response, wherein the composition administered in the first dose is the same as that administered second dose. In illustrative examples of this type, the first dose is administered to induce largely a cellular immune response to the target antigen, whereas the second dose is administered largely to elicit a humoral immune response to the target antigen. Upon completion of the administration steps of the strategy, both cellular and humoral immune responses develop to the target antigen. The two responses together thus provide effective or enhanced protection against a CMV infection or disease and/or condition that is transmitted by or otherwise associated with CMV.

In order to maximise the direct stimulation and activation of those CD4+CTLs that target the relevant gB polypeptide, the compositions used for the prime administration and the boost administration are, preferentially, the same.

### 6. Methods of Manufacture

The modified gB polypeptide is typically manufactured through recombinant expression methods. Such methods are well known in the art, and in particular methods of recombinant protein expression in mammalian cells (e.g., Chinese hamster ovary cells, CHO). In light of the heterologous signal peptide the recombinantly produced modified gB polypeptide is generally secreted from the host expressing cell into the culture supernatant.

Upon harvesting the supernatant (typically by centrifugation), the soluble fraction is purified by one or more of anionic exchange chromatography, cationic exchange chromatography, CHT type II chromatography, and HIC chromatography. In some preferred embodiments, the methods of producing the purified homotrimeric composition comprises a combination of anionic exchange chromatography, cationic exchange chromatography, and CHT type II chromatography In some embodiments, the purification of the trimeric compositions further comprises size exclusion chromatography.

In preferred embodiments, the modified gB polypeptide does not comprise a His tag, and therefore the purification methods do not comprise metal affinity chromatography.

### 7. Kits

The present invention also provides kits comprising an immunostimulatory composition as broadly described above and elsewhere herein. Such kits may additionally comprise alternative immunogenic agents for concurrent use with the immunostimulatory compositions of the invention.

In some embodiments, in addition to the immunostimulatory compositions of the present invention the kits may include suitable components for performing the prime-boost regimens described above. For example, the kit may include separately housed priming and boosting doses of the at least one polypeptide antigens.

The kits may comprise additional components to assist in performing the methods of the present invention such as, for example, administration device(s), buffer(s), and/or diluent(s). The kits may also include containers for housing the various components and instructions for using the kit components in the methods of the present invention.

In order that the invention may be readily understood and put into practical effect, particular preferred embodiments will now be described by way of the following non limiting examples.

### EXAMPLES

To develop a CMV vaccine, a modified gB polypeptide sequence was designed, based on the native HCMV AD169 strain gB protein (as set forth in SEQ ID NO: 1). The modified polypeptide was designed to possess a virion surface domain (as set forth in SEQ ID NO: 5) and an intravirion domain (as set forth in SEQ ID NO: 6). This amino acid sequence lacked at least a portion of the transmembrane region and the hydrophobic membrane proximal region, and the native furin cleavage site was removed through amino acid variation.

A portion of the transmembrane region of the native HCMV gB protein was deleted to facilitate protein secretion into cell culture medium. The furin protease cleaves gB into gp90 and gp58 subunits, which are covalently linked by disulfide bonds and the mature glycosylated gB obtains a trimeric form; the trimer subsequently dimerises as the protein assumes its final natural physical form in viral envelope. However, expression of HCMV gB with a furin cleavage site is shown to yield a lower concentration of monomeric form of gB protein. Therefore, it the present inventors hypothesised that making the furin cleavage site non-functional would enhance the protein production.

The modified gB polypeptide encoding nucleotide sequence was codon optimised to enhance protein expression in mammalian cells (SEQ ID NO: 9). To express the modified gB polypeptide, CHO-K1 cells were transfected with a mammalian expression plasmid encoding gB nucleotide sequence. Protein expression levels were confirmed in small scale shake flask cultures and fast stable pools were selected using Blasticidin and Zeocin.

The nucleic acid sequence encoding the modified gB polypeptide is identified in SEQ ID NO: 9.

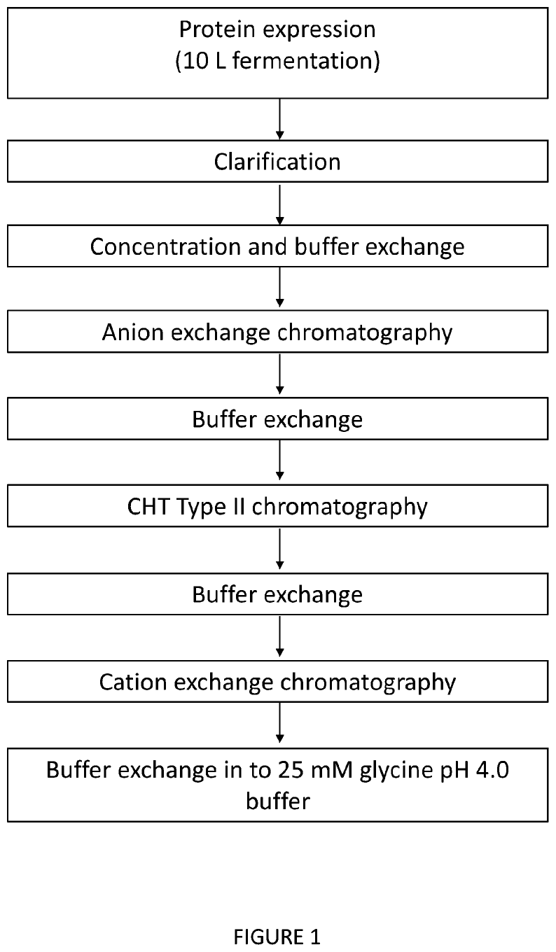

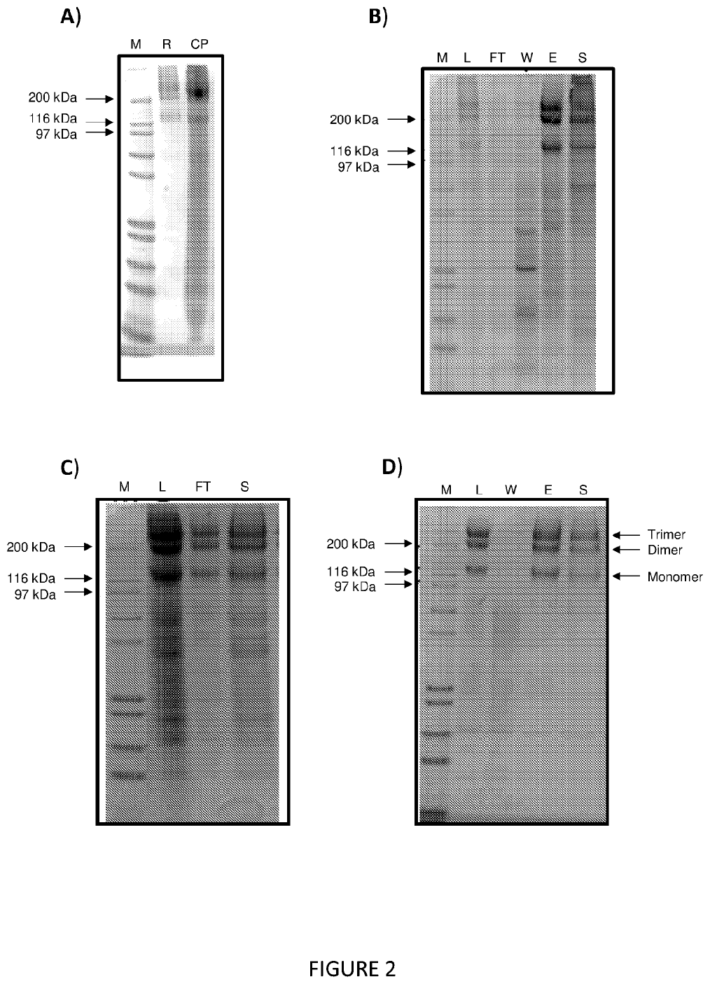

The CHO-K1 modified gB polypeptide clone expressing high levels of protein expression was scaled up and then used as a starting material for 10 L fermenter culture (FIG. 1). The fermentation process was stopped after day 12 and amount of modified gB polypeptide secreted into the supernatant was estimated using SDS-PAGE. The SDS-PAGE analysis revealed that high concentration of gB polypeptide secreted into cell supernatant (FIG. 2). However, in line with reference standard, expressed protein migrated at three different molecular sizes. This indicated that secreted gB polypeptide forms the multimeric form. The monomer, dimer and trimeric gB forms had approximate molecular mass of 150, 250 and 350 kDa respectively.

To purify the secreted gB polypeptide three different chromatography techniques, such as anion exchange, CHT Ceramic Hydroxyapatite Type II, and cation exchange chromatography columns were used (FIG. 2A-D). The final SDS-PAGE analysis indicates that multimers of gB polypeptide were present in the final purified sample and they migrate according to the molecular weight. If considering the multimer profile of the modified gB protein, SDS-PAGE analysis shows that the intensity of the gB protein staining at the approximate molecular weight corresponding to a gB trimer (i.e., at approximately 350 kDa) is significantly higher as compared to the staining at the molecular weight corresponding to a gB dimer (i.e., at 250 kDa) and the monomer (i.e., at 150 kDa). Collectively, these results suggest that expression of gB polypeptide extracellular and intracellular domains with a mutation at furin cleavage site in CHO-K1 cells can produce high concentration of gB trimer and a slightly reduced concentration of dimers and monomers.

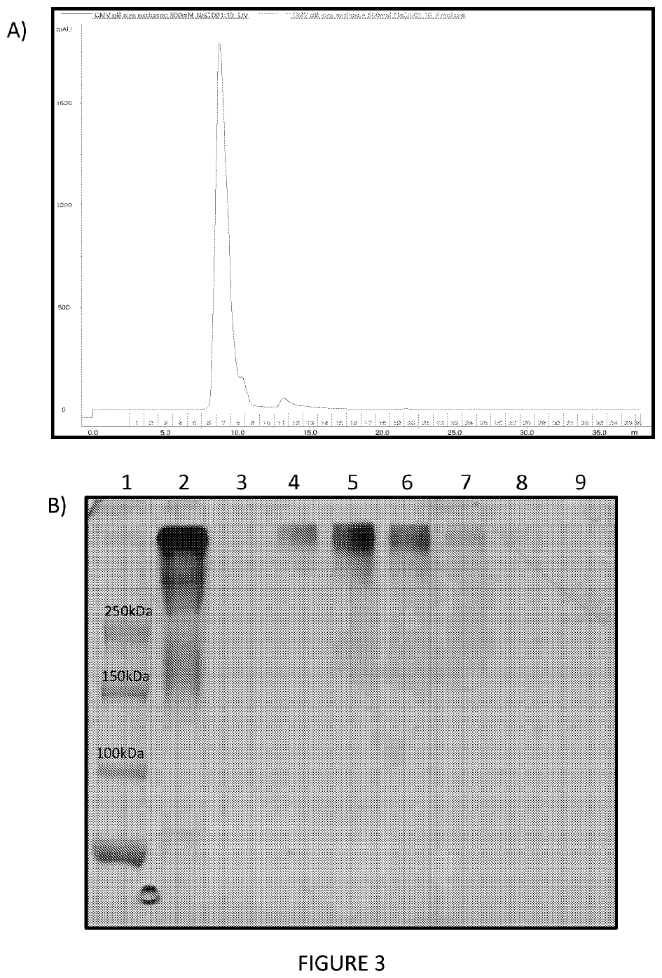

To characterise the gB polypeptide expressed in CHO cells was further analysed by size exclusion chromatography (FIG. 3A). The elution profile of the size exclusion indicated that gB polypeptide predominantly eluted as a single sharp peak, suggesting that gB likely to represent the prefusion trimeric form. Furthermore, the analysis of eluted fractions from the size exclusion chromatography on native SDS-PAGE gel also indicates that 90% of gB polypeptide present in the final purified sample was in trimeric form and only 10% of gB polypeptide seems to be as a monomer (FIG. 3B). If considering the multimer profile of gB, native SDS-PAGE analysis shows that intensity of gB trimer at higher molecular weight is significantly higher compared to dimer at 250 kDa and monomer at 150 kDa. Collectively, these results suggest that expression of gB polypeptide extracellular and intracellular domains with a mutation at furin cleavage site in CHO-K1 cells can produce high concentration of gB trimer and a slightly reduced concentration of dimers and monomers.

The recombinant HCMV gB vaccine with MF59 adjuvant used in phase II clinical trials achieved 50% protein protection in preventing HCMV infection in a population of HCMV-seronegative adolescent and postpartum women. The recombinant gB polypeptide used in these previous clinical trials was in monomeric form, but in naturally HCMV infected individuals the gB assumes its native prefusion trimeric conformation. Therefore, it is important to delineate which component of gB polypeptide is required for optimal induction of gB-specific antibody response.

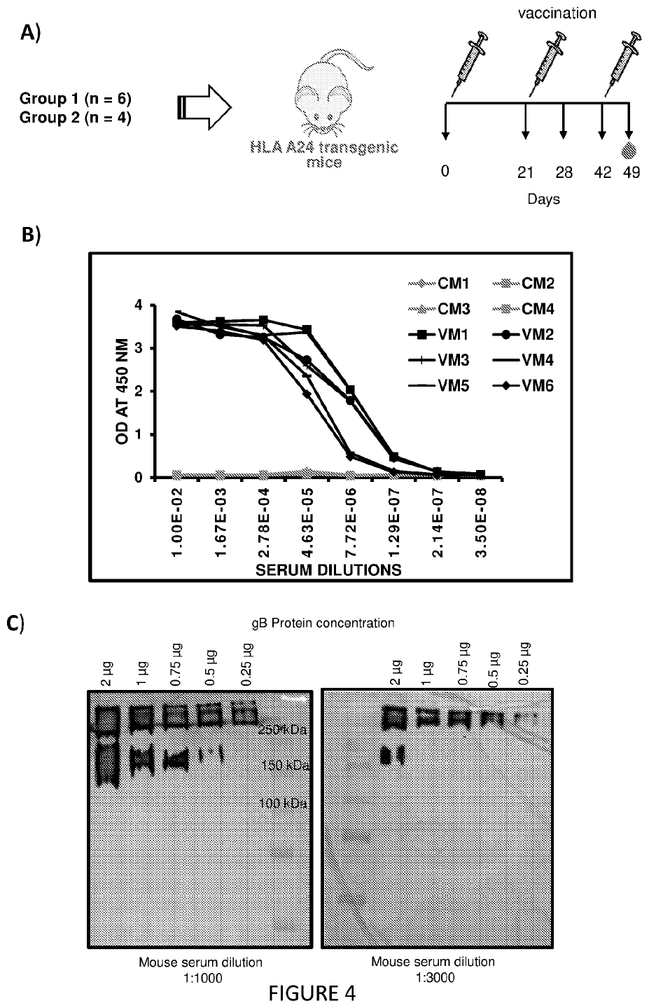

In order to determine the immunogenicity of three multimeric forms of gB polypeptide, human HLA A24 transgenic mice were immunised with the CMV vaccine formulated with CMV gB, CMVpoly and CpG1018 adjuvant on day 0, 21 and 42 or with Cpg1018 alone as a control formulation (FIG. 4A). The mice serum samples were collected after seven days of the third vaccine dose and then analysed for anti-gB antibody responses using ELISA. The sera obtained from mice immunised with CMV vaccine showed stronger binding to gB polypeptide (FIG. 4B).

The specificity of the gB antibody response was further characterised using Western blot analysis under non-reducing conditions to preserve the native prefusion conformation of gB polypeptide. Data obtained from the Western blot analysis indicated that sera from mice vaccinated with CMV vaccine strongly reacted to gB trimer, to a lesser extent to gB dimer, but no visible binding reaction was observed against gB monomer (FIG. 4C). Collectively these data suggest that to induce a strong antibody response against gB, it has to be formulated in its native trimeric confirmation.

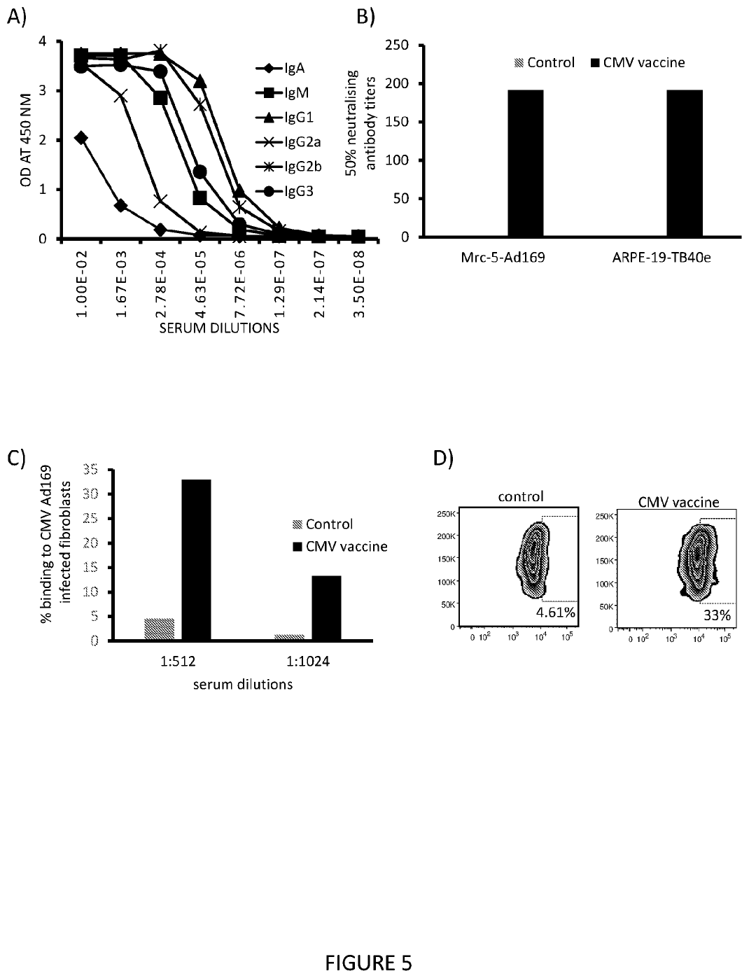

The isotypes of anti-gB antibodies in serum samples was also evaluated. The CMV vaccine induced robust gB-specific antibody response which included multiple isotypes, including IgA, IgM, IgG1 (Th2 like Ig isotype), and IgG2b, IgG2a, IgG3 (Th1 like Ig isotypes) (FIG. 5A) and in the functional microneutralisation assay gB-specific antibodies demonstrated strong neutralising ability against HCMV AD169 and TB40e strains Mrc-5 and ARPE-19 infection (FIG. 5B). Emerging evidence suggest that serum HCMV gB-specific IgG binding to cell-associated gB correlates with vaccine efficacy. In subsequent experiments, the ability of mouse serum antibodies binding to CMV AD169 infected fibroblasts was determined. It was found that serum antibodies from mice immunised with CMV vaccine formulated with native gB trimer, exhibited strong binding to cell-associated gB on fibroblasts infected with CMV AD169 strain compared to serum obtained from control mice (FIGS. 5C and 5D).

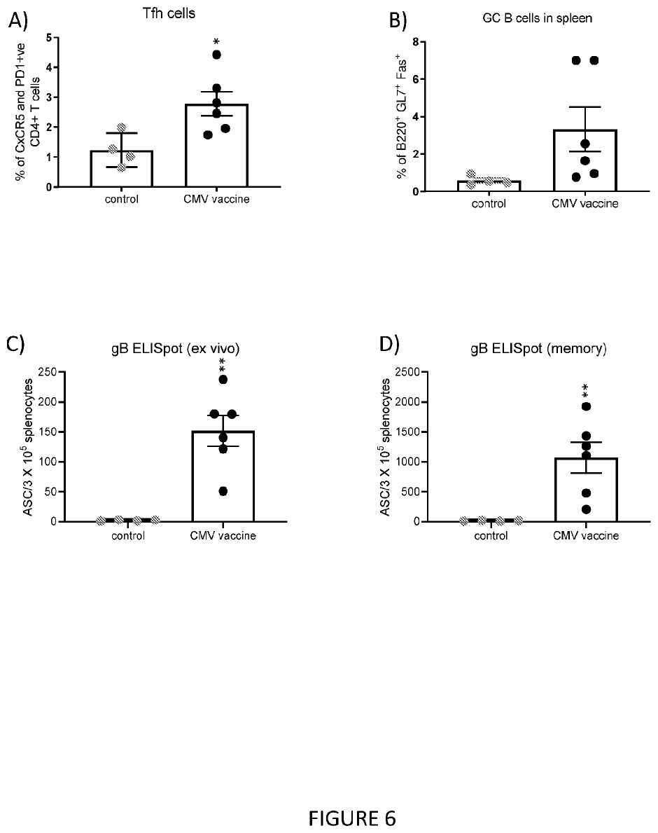

T follicular helper cells (TH) are a specialised subset of CD4⁺ T cells and play an important role in the formation of germinal centres (GCs). GCs are distinct structures that form within the B cell zones of secondary lymphoid organs during an ongoing immune response. B cells within GCs undergo rapid proliferation and antibody diversification, triggering the production different types of antibody isotypes, with greater affinity for their antigen targets. GCs are also the site where B cells can differentiate into antibody secreting plasma cells and memory B cells which allow long lasting antibody production. Thus, it is important to assess the T_{FH} and GCs B cell responses in vaccinated mice. The assessment of T_{FH} cell responses in spleen indicate that CMV vaccine with native gB trimer induces significantly higher T_{FH} cells compared to CpG1018 alone control (FIG. 6A). Further assessment of GC B cells indicated that the native gB in trimeric form induced significantly higher proportion of GC B cells (B220⁺GL7⁺Fas⁺) on day 49 compared to mice immunised with CpG 1018 alone (FIG. 6B). Finally, the analysis of CMV gB-specific IgG secreting plasma and memory B cells by ELISpot assay indicated that the CMV vaccine formulation with native trimeric gB induced a significantly higher plasma and robust memory B cell responses compared to CpG1018 alone immunisation (FIGS. 6C and 6D).

Evolving evidence suggests that HCMV-specific CD4⁺ T cells play a vital role in anti-viral immunity and in the potential maintenance of latently infected cells. In adults during primary infection with HCMV, CD4⁺ T cells are essential for the resolution of symptomatic disease. While in young children, persistent shedding of HCMV into urine and saliva is associated with a lack of HCMV specific CD4⁺ T cell response. In immunosuppressed solid organ transplant recipients, compromised HCMV-specific CD4⁺ T cells is associated with prolonged viremia and more severe clinical disease. In hematopoietic stem cell transplant recipients, it has been shown that HCMV-specific CD4⁺ T cells are required for HCMV-specific CD8⁺ T cells to exert their anti-viral effects. In addition, adoptive T-cell immunotherapy in transplant patients has revealed that the presence of HCMV-specific CD4⁺ T cells is required for the maintenance of HCMV-specific CD8⁺ T cells. These observations suggest that HCMV-specific CD4⁺ T cells play a crucial role in controlling the CMV infection and disease.

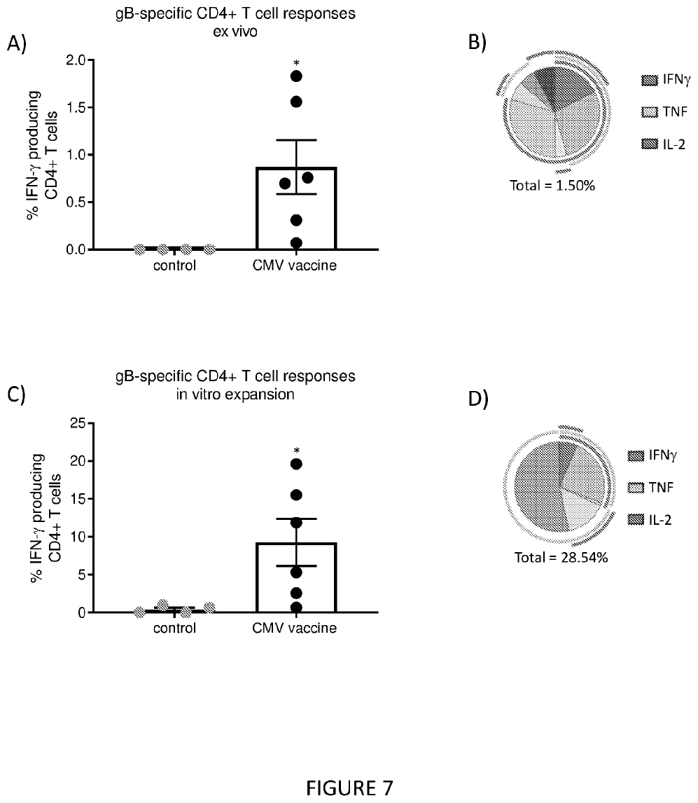

In subsequent experiments, the inventors delineated the ability of CMV vaccine formulated with native gB trimeric protein to induce CD4⁺ T cell responses. Interestingly, the CMV vaccine formulation with native gB trimer induced higher frequencies of IFN-γ producing trimeric gB-specific CD4⁺ T cell responses after ex vivo and a large proportion these cells were capable of inducing three cytokines (IFN-γ, IL-2 and TNF) or two cytokines (IFN-γ and TNF) simultaneously (FIGS. 7A and 7B). Additionally, in vitro stimulation of trimeric gB-specific CD4⁺ T cells with gB pepmix triggered rapid expansion of CD4⁺ T cells and a high proportion of CMV gB-specific CD4⁺ T also showed their capacity to secrete three cytokines (IFN-γ, TNF, IL-2) or two cytokine combinations (IFN-γ and TNF or TNF and IL-2) simultaneously (see, FIGS. 7C and 7D). This suggests that native gB trimer can induce strong memory immune response.

Collectively, these results indicate that a novel modified gB polypeptide trimer can be produced without the inclusion of any mutations or a linker such as (Gly4Ser) 3. The present invention discloses a novel approach for the development of a modified gB polypeptide trimer by removing at least a portion of the transmembrane domain and virion surface domain of the native full-length HCMV gB protein. The modified gB polypeptide trimer can be purified to homogeneity using anion exchange, CHT type II, cation exchange and size exclusion chromatography techniques. The CMV vaccine formulated with native gB trimer induces strong antibody and neutralising antibody responses against multiple HCMV strains. However, for the first time it is shown that antibodies induced following CMV vaccine, predominantly react to the modified gB polypeptide trimer and these antibodies also capable binding to gB protein expressed on fibroblast infected with the HCMV AD169 strain. Furthermore, although previous studies are confined to assess gB-specific neutralising antibody responses, the inventors revealed that the novel CMV vaccine formulation with the modified prefusion gB polypeptide trimer (as confirmed by cryo-EM, data not shown) demonstrates a substantial ability to induce robust T_{FH} cell, GCs, B cells, antibody secreting plasma, memory B cells and CD4⁺ T cell responses.

### Materials & Methods

### Modified gB Polypeptide Expression and Purification.

The coding sequence of HCMV gB from HCMV strain AD169, was codon optimised for mammalian expression to enhance the protein expression and cloned into a mammalian expression vector. The native N-terminal signal sequence (i.e., amino acids 1 to 31 of the sequence set forth in SEQ ID NO: 1) was replaced with the heterologous IgG heavy chain signal peptide to secrete the expressed polypeptide into cell culture supernatant. The modified gB polypeptide DNA sequence encodes the amino acid sequence set forth in SEQ ID NO: 7. The encoded sequence of the furin cleavage site was mutated from Arg456 to Gln, Arg458 to Thr and Arg459 to Gln (corresponding the residue numbering set forth in SEQ ID NO: 1). Chinese Hamster Ovary (CHO) K1 cells were transfected with the modified gB polypeptide plasmid and stable cells expressing modified gB polypeptide were selected with 9 μg/mL blasticidin and 400 μg/mL Zeocin. Modified gB polypeptide was expressed in a 10 L bioreactor in fed-batch mode. On day 12, cell culture was harvested and centrifuged to separate supernatant from cell debris. The supernatant was buffer exchanged into 200 mM Tris-HCL (pH 8.0) and then loaded on Poros 50HQ resin (anion exchange chromatography). The column was washed with 20 mM Tris-HCL, 70 mM NaCl (pH 8.0) buffer, before eluting the protein with 20 mM Tris-HCL, 180 mM NaCl (pH 8.0) buffer. Eluted polypeptide was buffer exchanged with 5 mM phosphate buffer (pH 7.0) and passed through a ceramic hydroxyapatite (CHT) Type II to eliminate host cell protein contaminates. To further improve the purity, the modified gB polypeptide was buffer exchanged into 50 mM sodium acetate (NaOAc) (pH 5.0) buffer and then loaded on POROS XS (cation exchange chromatography) column. The modified gB polypeptide bound to the POROS XS column was eluted with 50 mM NaOAc, 500 mM NaCl (pH 5.0) buffer and then buffer exchanged against 25 mM glycine (pH 4.0) buffer. The final purified polypeptide concentration was determined by UV280 using extinction coefficient 1.209, analysed on SDS-PAGE gel and stored at −70° C.

### Mouse Immunisation.

Human HLA transgenic mice (HLA A24) were immunised with the modified gB polypeptide (5 μg) and CMVpoly20PLNH (30 μg) formulated with CpG1018 adjuvant (50 μg) on day 0. The control group mice were injected with CpG1018 (50 μg) alone. On day 21 and day 42, mice were tail bled and boosted with identical vaccine or control formulations. On day 49, mice were euthanised and serum was collected to assess HCMV gB-specific antibody response.

### Enzyme-Linked Immunosorbent Assay.

Anti-gB antibody titres were measured using an enzyme-linked immunosorbent assay (ELISA). Polystyrene 96-well half-area plates were coated overnight with 5 μg/mL of HCMV gB polypeptide diluted in phosphate buffer saline (PBS). Plates were incubated at 4° C. overnight. Unbound modified gB polypeptide was washed and then plates blocked with 5% skim milk powder in PBS. Six-fold serial dilutions of serum samples in PBS 5% skim milk powder were performed. Serially diluted serum samples were added to the plates and incubated at room temperature. Plates were washed and treated with HRP-conjugated goat anti-mouse Ig secondary antibody for 1 hour at room temperature. Plates were washed and 3,3′5,5′-tetramethylbenzidine substrate was added for colour development. The colour development reaction was quenched by adding 1N HCL and the absorbance at 450 nM was determined using ELISA reader.

### Western Blot Analysis.

To determine the most immunogenic form of modified gB polypeptide a Western blot analysis was performed. The multimeric forms of modified gB purified polypeptide were separated on 8% SDS-PAGE under non-reducing conditions. Two different 8% SDS-PAGE gels were run simultaneously with five different gB polypeptide concentrations (ranging from 2 to 0.25 μg). Following polypeptide resolution on SDS-PAGE, the polypeptide was transferred to Hybond-C nitrocellulose membrane. After transfer, membranes were washed, blocked and the probed with two different concentrations (1:1000 and 1:3000) of mouse serum. Membranes were washed and then incubated with HRP-conjugated goat anti-mouse Ig antibody, followed by a wash and incubation with Immobilon ECL ultra western HRP substrate. The signal was captured using Invitrogen CL1500 Chemi Gel Doc System.

### Mouse IgG ELISpot Assay.

To measure ex vivo gB-specific antibody secreting cells, PVDF ELISpot plates (Millipore) were treated with 70% ethanol. Plates were washed five times with distilled water, coated with 100 μL/well HCMV gB protein (25 μg/mL) or anti-IgG antibody (15 μg/mL) as a positive control and incubated overnight at 4° C. Plates were blocked with DMEM containing 10% serum, 300,000 cells/well in triplicates from each mouse was added and then incubated for 18 hours in a 37° C. humidified incubator with 5% CO₂. Cells were removed and plates were washed. Detection antibody anti-IgG conjugated to HRP (MABTECH) was added and incubated for 2 hours at room temperature. Plates were washed; Streptavidin-ALP was added and incubated at room temperature for 1 hour followed by washing and treating plates with substrate solution containing BCIP/NBT (Sigma-Aldrich) until colour development is prominent. Colour development was stopped by washing plates with water and plates were kept for drying overnight. To measure memory B cell response, the spleen cells (5×10⁵) were activated with a mixture of R484 and recombinant mouse IL-2 for five days in 24 well plate and then ELISpot was carried out as stated above. Number of spots were counted in an ELISpot reader.

### Microneutalisation Assay.

Neutralising activity was determined against AD169 and TB40/E strains of HCMV. Human fibroblast Mrc-5 or Adult Retinal Pigment Epithelial (ARPE-19) cells were plated in 96-well flat-bottomed plates. The next day, serum samples collected on day 49 from mice vaccinated with CMV vaccine or control formulations were serially diluted and added to a standard number of virus particles (1000 p.f.u. per well) diluted in DO (DMEM with no serum) in 96-well U-bottomed plates and incubated for 2 h at 37° C. and 5% CO₂. As a positive control, virus without serum and a negative-control serum without virus were also included in the test. The serum/CMV mixture was then added to the Mrc-5 or ARPE-19 cells and incubated at 37° C. and 5% CO₂ for 2 hours. After incubation, the mixture was discarded and the cells washed gently five times with DMEM containing 10% FCS (D10) and a final volume of 200 mL R10 was added to each well, followed by incubation for 16-18 hours at 37° C. and 5% CO₂. The cells were fixed with 100 mL chilled methanol and incubated with Peroxidase Block (Dako) followed by mouse anti-CMV IE-1/IE2 mAb (Chemicon) at room temperature for 3 hours. Cells were then incubated with 50 mL HRP-conjugated goat anti-mouse Ig (diluted 1:200 in PBS) per well for 3 hours at room temperature. The cells were stained with 20 μL diaminobenzidine plus substrate (Dako) per well for 10 min at room temperature and positive nuclei that stained dark brown were counted. The neutralising titre was calculated as the reciprocal of the serum dilution that gave 50% inhibition of IE-1/IE-2-expressing nuclei.

CMV gB-Specific Antibodies Binding to Cell-Associated gB on CMV-Infected Fibroblasts Assay.

Human fibroblasts cell line, Mrc-5 cells were grown to 50% confluency in a T75 flask. Cell were infected with CMV AD169 strain at multiplicity of infection (MOI) of 2.0 at 37° C. and 5% CO₂ for 2 hours. Following infection cells were washed and incubated with DMEM containing 10% FCS for 48 hours to allow cell-cell virus spared. Infected cells were washed with PBS and then cells were dislodged with trypsin-EDTA. Cells were washed, counted and then resuspended at 106 viable cells/mL. Cells were stained with cell trace violet and incubated for 20 minutes at room temperature. Cells were washed and then fixed with 4% paraformaldehyde for 10 minutes at room temperature. Cells were washed twice, plated 20,000/well in 96-well V-bottom plates, cells were pelleted by centrifugation and supernatant was discarded. Mouse serum samples obtained from HLA A24 human transgenic mice following immunisation with CMV vaccine or place on day 49 were polled and then diluted to 1:512 or 1:1024. Diluted serum samples were added to the Mrc-5 cells and incubated for 2 hours at 37° C. and 5% CO₂. Cells were washed and then stained with anti-mouse AF488 IgG (H+L) for 30 minutes at 4° C. Cells were acquired on a BD FACSCanto II and data was analysed using FlowJo software (Tree Star). The percentage of CMV gB-specific antibody binding to CMV infected fibroblasts was calculated from the percentage of viable AF 488 positive cells.

### Intracellular Cytokine Staining to Assess IFN-γ, Multiple Cytokine, Germinal Centre B Cells or T Follicular Helper Cell Responses.

To detect HCMV gB-specific CD4⁺ T cell responses following vaccination, splenocytes were stimulated with 0.2 μg/mL of gB pepmix (gB overlapping peptides-15mers with 11 amino acid overlap) in the presence of GolgiPlug® (BD PharMingen) for 6 hours, cells were washed twice, then incubated with APC-conjugated anti-CD3, FITC-conjugated anti-CD4 and PerCP conjugated anti-CD8. Cells were fixed and permeabilised using a BD Cytofix/Cytoperm kit, then incubated with PE conjugated anti-IFN-γ. To assess the expression of multiple cytokines, cells were stained with PerCP conjugated anti-CD8 and BV786 anti-CD4 surface markers and then intracellularly with PE-conjugated anti-IFN-γ, PE-Cy7 conjugated anti-TNF, FITC conjugated CD017a and APC conjugated anti-IL-2. To assess the germinal centre B cell response splenocytes or cells from lymph nodes from vaccinated mice were stained with PE conjugated anti-B220, FITC conjugated anti-GL7 and APC conjugated anti-CD95. To assess the T follicular helper cells (T_{FH}) cell response splenocytes from vaccinated mice were stained with PerCP conjugated anti-CD8, BV786 anti-CD4, CxCR5 and PD-1 surface markers. Cells were acquired on a BD FACSCanto II and data was analysed using FlowJo software (Tree Star).

In Vitro Expansion of CMV-Specific CD4⁺ and CD8⁺ T Cells Following Vaccination.

Following vaccination, 5×10⁶ splenocytes from immunised mice were isolated and stimulated with 0.2 μg/mL HCMV gB pepmix (gB overlapping peptides-15mers with 11 amino acid overlap) and cell were cultured in a 24 well plate for 10 days at 37° C. 10% CO₂. Cultures were supplemented with recombinant IL-2 on days 3 and 6 and on day 10 and T cell specificity was assessed using ICS assay.

### Example 2

### Negative-Stain Method and Observation of Gb Homotrimers

To observe gB of HCMV via negative-stain electron microscopy the protein was purified by size-exclusion chromatography (Cytiva S200 10/300) in gel filtration buffer (50 mM Tris pH 7.2, 150 mM NaCl or 25 mM Tris pH 7.1, 500 mM NaCl) and concentrated to 1.8 or 3 mg/mL. The protein was diluted 1000-fold and applied to glow-discharged carbon-coated Formvar grids, stained with 2% (w/v) uranyl acetate for two minutes, then blotted and air-dried for 10 minutes. Samples were imaged using FEI Tecnai F30 G2 TEM or JEOL JEM 1011 TEM.

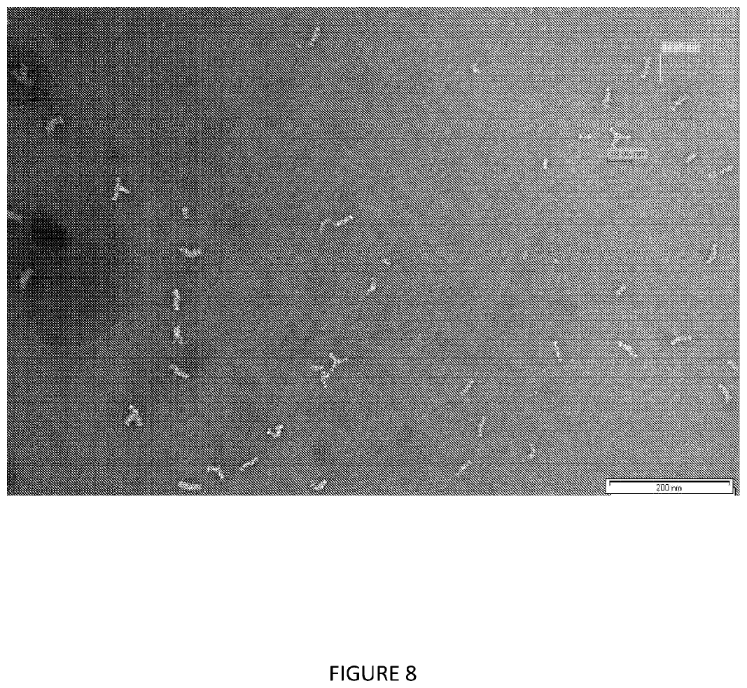

From the preliminary data collected it appears the protein is pure and forms complexes of clear homotrimers (FIG. 8). These images show different views (side, top and bottom) of the trimeric complexes. The protein is very stable and based on this analysis we can bypass a lot of optimising on negative-stain and analyse using cryo-EM to generate 2D and 3D class averages to possibly attain atomic, or near-atomic, resolution.

The disclosure of every patent, patent application, and publication cited herein is hereby incorporated herein by reference in its entirety.

The citation of any reference herein should not be construed as an admission that such reference is available as “prior art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Cui B, Liu X, Fang Y, Zhou P, Zhang Y, Wang Y. Flagellin as a vaccine adjuvant. Expert Rev Vaccines. 2018 April; 17 (4): 335-349. doi: 10.1080/14760584.2018.1457443. Epub 2018 Mar. 30. PMID: 29580106.
Fu L Y, Bonhomme L A, Cooper S C, Joseph J G, Zimet G D. Educational interventions to Increase HPV vaccination acceptance: a systematic review. Vaccine. 2014 Apr. 7; 32 (17): 1901-20. doi: 10.1016/j.vaccine.2014.01.091. Epub 2014 Feb. 14. PMID: 24530401; PMCID: PMC4285433.
Pass R F. Development and evidence for efficacy of CMV glycoprotein B vaccine with MF59 adjuvant. J Clin Virol. 2009 December; 46 Suppl 4 (Suppl 4): S73-6. doi: 10.1016/j.jcv.2009.07.002. Epub 2009 Jul. 31. PMID: 19647480; PMCID: PMC2805195.
Pass R F, Zhang C, Evans A, Simpson T, Andrews W, Huang M L, Corey L, Hill J, Davis E, Flanigan C, Cloud G. Vaccine prevention of maternal cytomegalovirus infection. N Engl J Med. 2009 Mar. 19; 360 (12): 1191-9. doi: 10.1056/NEJMoa0804749. PMID: 19297572; PMCID: PMC2753425.
Spaete R R. A recombinant subunit vaccine approach to HCMV vaccine development. Transplant Proc. 1991 June; 23 (3 Suppl 3): 90-6. PMID: 1648843.

What is claimed is:

1. A composition comprising isolated homotrimers of a modified gB polypeptide.

2. The composition of claim 1, wherein the modified gB polypeptide comprises an amino acid sequence that corresponds to a human cytomegalovirus (HCMV) glycoprotein B (gB), wherein the modified gB polypeptide lacks at least a portion of the transmembrane domain.

3. The composition of claim 2, wherein the transmembrane domain corresponds to amino acid residues 751 to 771 of the native full-length polypeptide sequence set forth in SEQ ID NO: 1.

4. The composition of any one of claims 1 to 3, wherein the modified gB polypeptide further comprises an N-terminal signal peptide.

5. The composition of claim 4, wherein the signal peptide results in the secretion of the modified gB polypeptide from a cell.

6. The composition of claim 4 or claim 5, wherein the signal peptide is derived from an immunoglobulin isotype.

7. The composition of claim 6, wherein the immunoglobulin isotype is selected from any one of IgA, IgD, IgE, IgG, and IgM.

8. The composition of any one of claims 4 to 7, wherein the signal peptide comprises, consists, or consists essentially of, the amino acid sequence set forth in SEQ ID NO: 7.

9. The composition of any one of claims 1 to 7, wherein the modified gB polypeptide comprises a first region that corresponds to at least a portion of the native gB protein virion surface domain; and a second region that corresponds to at least a portion of the native gB protein intravirion domain.

10. The composition of claim 9, wherein the native gB protein virion surface domain comprises the amino acid sequence set forth in SEQ ID NO: 5.

11. The composition of claim 9 or claim 10, wherein the gB protein virion surface domain does not comprise the hydrophobic membrane-proximal region.

12. The composition of any one of claims 9 to 11, wherein the gB protein intravirion domain comprises the amino acid sequence set forth in SEQ ID NO: 6.

13. The composition of any one of claims 1 to 12, wherein the polypeptide does not comprise a furin cleavage site motif.

14. The composition of any one of claims 1 to 13, wherein the amino acid residue corresponding to position 456 of the wild-type HCMV gB is an amino acid other than arginine.

15. The composition of any one of claims 1 to 14, wherein the amino acid residue corresponding to position 456 of the wild-type HCMV gB is glutamine or threonine.

16. The composition of any one of claims 1 to 15, wherein the amino acid residue corresponding to position 458 of the wild-type HCMV gB is an amino acid other than arginine.

17. The composition of any one of claims 1 to 16, wherein the amino acid residue corresponding to position 458 of the wild-type HCMV gB is threonine or glutamine.

18. The composition of any one of claims 1 to 17, wherein the amino acid residue corresponding to position 459 of the wild-type HCMV gB is an amino acid other than arginine.

19. The composition of any one of claims 1 to 18, wherein the amino acid residue corresponding to position 459 of the wild-type HCMV gB is glutamine or threonine.

20. The composition of claim any one of claims 1 to 19, wherein the amino acid sequence comprises, consists, or consists essentially of the amino acid sequence set forth in SEQ ID NO:4.

21. The composition of any one of claims 1 to 20, wherein the trimeric modified gB polypeptide complexes form dimers.

22. The composition of any one of claims 1 to 21, wherein the modified gB polypeptide enhances immunogenicity as compared to native gB polypeptide.

23. A nucleic acid composition that encodes the modified gB polypeptide defined in any one of claims 1 to 22.

24. An expression vector encoding the nucleic acid of any one claim 23, operably linked to a regulatory element.

25. A cell comprising the expression vector of claim 24.

26. A pharmaceutical composition comprising a substantially homogenous preparation of modified gB polypeptide in trimeric form; and a pharmaceutically acceptable, carrier, diluent and/or excipient.

27. The composition of claim 26, wherein the modified gB polypeptide comprises an amino acid sequence that corresponds to a human cytomegalovirus (HCMV) glycoprotein B (gB), wherein the modified gB polypeptide lacks at least a portion of the transmembrane domain.

28. The composition of claim 27, wherein the transmembrane domain corresponds to amino acid residues 751 to 771 of the native full-length polypeptide sequence set forth in SEQ ID NO: 1.

29. The composition of any one of claims 26 to 28, wherein the modified gB polypeptide further comprises an N-terminal signal peptide.

30. The composition of claim 29, wherein the signal peptide results in the secretion of the modified gB polypeptide from a cell.

31. The composition of claim 29 or claim 30, wherein the signal peptide is derived from an immunoglobulin isotype.

32. The composition of claim 31, wherein the immunoglobulin isotype is selected from any one of IgA, IgD, IgE, IgG, and IgM.

33. The composition of any one of claims 29 to 32, wherein the signal peptide comprises, consists, or consists essentially of, the amino acid sequence set forth in SEQ ID NO: 7.

34. The composition of any one of claims 26 to 33, wherein the modified gB polypeptide comprises a first region that corresponds to at least a portion of the native gB protein virion surface domain; and a second region that corresponds to at least a portion of the native gB protein intravirion domain.

35. The composition of claim 34, wherein the native gB protein virion surface domain comprises the amino acid sequence set forth in SEQ ID NO: 5.

36. The composition of claim 34 or claim 35, wherein the gB protein virion surface domain does not comprise the hydrophobic membrane-proximal region.

37. The composition of any one of claims 34 to 36, wherein the gB protein intravirion domain comprises the amino acid sequence set forth in SEQ ID NO: 6.

38. The composition of any one of claims 26 to 37, wherein the polypeptide does not comprise a furin cleavage site motif.

39. The composition of any one of claims 26 to 38, wherein the amino acid residue corresponding to position 456 of the wild-type HCMV gB is an amino acid other than arginine.

40. The composition of any one of claims 26 to 39, wherein the amino acid residue corresponding to position 456 of the wild-type HCMV gB is glutamine or threonine.

41. The composition of any one of claims 26 to 40, wherein the amino acid residue corresponding to position 458 of the wild-type HCMV gB is an amino acid other than arginine.

42. The composition of any one of claims 26 to 41, wherein the amino acid residue corresponding to position 458 of the wild-type HCMV gB is threonine or glutamine.

43. The composition of any one of claims 26 to 42, wherein the amino acid residue corresponding to position 459 of the wild-type HCMV gB is an amino acid other than arginine.

44. The composition of any one of claims 26 to 43, wherein the amino acid residue corresponding to position 459 of the wild-type HCMV gB is glutamine or threonine.

45. The composition of claim any one of claims 26 to 44, wherein the amino acid sequence comprises, consists, or consists essentially of the amino acid sequence set forth in SEQ ID NO: 4.

46. The composition of any one of claims 26 to 45, wherein the trimeric modified gB polypeptide complexes form dimers.

47. The composition of any one of claims 26 to 46, wherein the modified gB polypeptide enhances immunogenicity as compared to native gB polypeptide.

48. The composition of any one of claims 26 to 47, further comprising at least one CMV T cell epitope.

49. The composition of any one of claims 26 to 48, further comprising an adjuvant.

50. A method of treating or preventing a CMV-associated disease in a subject, the method comprising administering to the subject an effective amount of the composition of any one of claims 1 to 22 or 26 to 49.

51. The use of a composition of any one of claims 1 to 22 in the manufacture of a medicament for treatment or prevention of a CMV-associated disease.

52. The composition of claim 51, wherein the CMV-associated disease is a cancer.

53. The use of a composition of any one of claims 1 to 22 in the manufacture of a medicament for the treatment or prevention of a CMV infection.
